(19) United States
(12) Patent Application Publication
(10) Pub. No.: US 2026/0306383 A1
(43) Pub. Date: Oct. 8, 2026

(54) FLUORINE-FREE DIARYLETHENE PHOTOCHROMIC COMPOUND SYSTEM FOR REVERSIBLE HAIR COLORATION

(71) Applicants: The Procter & Gamble Company, Cincinnati, OH (US); School of Materials Science and Engineering, Nanyang Technological University, Singapore (SG)

(72) Inventors: Shufen FAN, Singapore (SG); Mei Yi TEE, Singapore (SG); Sung Ju CHO, Singapore (SG); Rui Andre GONÇALVES CARDOSO, Singapore (SG); Yeng Ming LAM, Singapore (SG); Teddy SALIM, Singapore (SG)

(21) Appl. No.: 19/632,581

(22) Filed: Mar. 30, 2026

Related U.S. Application Data
(60) Provisional application No. 63/783,536, filed on Apr. 4, 2025.

(51) Int. Cl. A61K 8/49; A45D 19/02; A45D 34/00; A45D 34/04; A61Q 5/06

(52) U.S. Cl. CPC A61K 8/4986; A45D 19/024; A45D 19/026; A45D 34/045; A61Q 5/06; A45D 2034/002; A45D 2200/205; A61K 2800/30; A61K 2800/438; A61K 2800/87

(57) ABSTRACT

This disclosure relates to fluorine-free photochromic compounds with controllable color upon activation, rapid activation to a colored state, persistent activated color in ambient lighting, and chemical compatibility common cosmetic solvents and other cosmetic formulation components. The photochromic compounds include a fluorine-free bridge with pendant heterocycles and substituted aromatic groups to provide the favorable combination of properties. The photochromic compounds can used to reversibly or irreversibly color hair or other substrates such as skin and textiles. Coloring compositions including multiple different photochromic compounds can be used to provide a tunable activated color based on selective activation or deactivation of individual photochromic compounds. The disclosure further provides related methods and products using or incorporating the photochromic compounds for coloring a substrate.

### FIELD OF THE INVENTION

The disclosure relates to fluorine-free photochromic compounds with controllable color upon activation, rapid activation to a colored state, persistent activated color in ambient lighting, and chemical compatibility common cosmetic solvents and other cosmetic formulation components. The photochromic compounds include a fluorine-free bridge with pendant heterocycles and substituted aromatic groups. The photochromic compounds can used to reversibly or irreversibly color hair or other substrates such as skin, other keratinous tissues, mucous membranes, mucocutaneous tissues, and synthetic and natural fibers, textiles, and surfaces.

### BACKGROUND OF THE INVENTION

Altering the color of human hair has long been used to cosmetically adapt a person's appearance to the fashion and style trends of the day. Conventional hair coloring techniques typically involve the application of a temporary, semi-permanent, demi-permanent or permanent chemical dye (oxidative or non-oxidative) to the hair to achieve a color change. Conventional hair dyes can provide a wide variety of hair color changes, hair lightening effects and hair darkening effects. However, conventional hair dyes are limited to providing a single hair color change. Additional hair color changes require application of a different hair dye composition. If a user desires a return to their natural hair color, then they must wait for the dye to be washed out or, in the case of permanent dyes, for the hair to grow out and the dyed portion cut off. In some cases, a bleaching process may be required to remove color. Thus, the use of conventional hair dyes can be inconvenient and time consuming.

To address some of the drawbacks of conventional hair dyes, photochromic dyes have been examined for color change options and flexibility. For example, FR 1,604,929 describes a hair composition that contains compounds such as nitrobenzylpyridines, thiosemicarbazones or spiropyran derivatives. When these compounds are exposed to electromagnetic radiation (“EMR”) of a particular wavelength (e.g., ultraviolet radiation with a wavelength of 100-400 nm), the dye compound undergoes a conformational change that alters the color of the dye. When the EMR is removed, the molecule reverts to its previous conformation and color. Compositions that contain the photochromic dyes disclosed in FR 1,604,929 may provide additional flexibility and convenience for changing hair or skin color, but it would be desirable to provide a hair coloring composition that does not depend on the presence or absence of a particular wavelength of EMR to maintain the desired color change.

In another example, U.S. Pat. No. 6,123,952 discloses a cosmetic composition that contains a thermally-irreversible, photochromic coloring agent, preferably selected from diarylethene compounds and fulgide compounds. According to U.S. Pat. No. 6,123,952, after the composition is applied to a bodily surface, it is exposed to EMR of a particular wavelength (e.g., ultraviolet radiation) that causes the photochromic dye to change color, thereby imparting a color change to the bodily surface. However, when the UVR is removed the photochromic dye retains its color. When the dye is exposed to EMR having a particular wavelength that is different from the previous EMR, the dye returns to its original color. While the hair dye compositions of U.S. Pat. No. 6,123,952 may provide additional flexibility and convenience for coloring hair or skin, these compositions are still limited to a single hair color change (i.e., coloring and de-coloring).

Another disadvantage of photochromic dyes, such as those disclosed in U.S. Pat. No. 6,123,952, is the lack of suitable solvents for use in hair dye compositions. For example, p-type photochromic dyes such as diarylethenes are readily dissolved in organic solvents such as toluene or formaldehyde. However, these types of organic solvents are generally not suitable for cosmetic use on skin and hair. While U.S. Pat. No. 6,123,952 discloses solvents that may be suitable for lotions and creams, these solvents may not be suitable for hair dye compositions.

Existing commercial diarylethene (DAE) materials that belong to the class of p-type photochromic materials are used in niche applications such as photo switches, which require the materials to have very fast color switching on/off speed and durability over tens of thousands of cycles, while remaining thermally stable. To provide these characteristics, traditional DAE materials are designed with a perfluorinated bridge such as a perfluoro cyclopentene backbone. However, perfluoro cyclopentene is a type of polyfluoroalkyl substance (PFAS) having C—F bonds that cannot be broken down easily and which can persist in the environment indefinitely. Furthermore, these DAE materials were designed mainly for specific usages in optoelectronics, and their color stability when exposed to mixed lighting (e.g., UV+white light/ambient indoor lighting) may be low, such that their color fades too quickly in a mixed lighting condition. Additionally, these DAE materials are often incompatible with acceptable and common solvents used for consumer goods products.

US2021/0308020 describes using a thermochromic encapsulated liquid crystal to provide a tunable hair color change. In particular, the publication describes how the encapsulated liquid crystal can be tuned to provide a static color effect by using a broader bandwidth such that upon reaching the starting point a color is observed and the clearing point is above the temperature that the composition is likely to encounter. However, the drawback with thermochromic dyes is the dependence on a particular temperature or temperature range to provide a color change or multiple color changes. Thus, the options for a user to tune their hair color with a thermochromic dye may be limited relative to a photochromic dye.

Additionally, with conventional hair dye products, difficulties have been encountered in providing consistent color effects to hair of different colors (e.g., blond, brown and black hair). To overcome this issue, medium and darker colored hair are commonly pre-treated to lighten the color of the hair (e.g., bleaching) and then applying the desired finish color.

### SUMMARY OF THE INVENTION

Accordingly, it would be desirable to provide photochromic compounds and related coloring compositions that are fluorine-free, yet provide favorable color and color-change characteristics for substrates such as hair, skin, other keratinous or keratin-containing tissues, mucous membranes, mucocutaneous tissues, and synthetic and natural fibers, textiles, and surfaces in various coloring applications. It would also be desirable to provide photochromic compounds and coloring compositions that that enable a user to impart a stable color to a substrate that fades slowly when exposed to mixed/ambient lighting (reversible color change) or a stable color than maintains its colored state indefinitely (irreversible color change). It would also be desirable to provide photochromic compounds and coloring compositions that that enable a user to conveniently change their hair color multiple times with a single hair coloring composition. It would also be desirable to provide a photochromic coloring composition that contains a solvent suitable for use on human skin and hair. It would also be desirable to provide a lineup of tunable coloring products, for example which provide a consistent hair color change to users with different hair colors without the need to pretreat darker colored hair. It would also be desirable for the product lineup to enable users with different hair colors to conveniently change their hair color multiple times with a single hair dye composition.

In an aspect, the disclosure relates to a product for reversibly or irreversibly coloring a substrate (e.g., keratinous or textile substrate), the product comprising: a container; a coloring composition in the container, the coloring composition comprising a photochromic compound comprising at least one of: (A) a fluorine-free first compound comprising (1) a cyclic bridge comprising one ethylenically unsaturated bond, (2) a first substituent comprising a first unsaturated heterocycle bonded to a first carbon atom in the ethylenically unsaturated bond and a first aromatic group or a first halogen bonded to the first unsaturated heterocycle, and (3) a second substituent comprising a second unsaturated heterocycle bonded to a second carbon atom in the ethylenically unsaturated bond and a second aromatic group bonded to the second unsaturated heterocycle; (B) a fluorine-free second compound comprising an activated analog of the fluorine-free first compound; and (C) a fluorine-free third compound comprising a dyotropic rearrangement of the fluorine-free first compound (or of the fluorine-free second compound); a light source adapted to provide one or both of activating electromagnetic radiation (EMR) having a wavelength of 250 nm to 395 nm and deactivating EMR having a wavelength of 400 nm to 1400 nm.

### BRIEF DESCRIPTION OF THE DRAWINGS

FIG. 1 illustrates a photochromic compound according to the disclosure in an unactivated base state (left), an activated analog state (middle), and a dyotropic rearrangement state (right) after application of electromagnetic radiation (EMR).

FIG. 2 illustrates a synthesis method for forming a photochromic compound according to the disclosure.

FIG. 3 illustrates a synthesis method for forming a photochromic compound according to the disclosure.

FIG. 4 is a graph illustrating absorption intensity vs. activation time for a photochromic compound according to the disclosure.

FIG. 5 is a graph illustrating absorption intensity vs. deactivation time for a photochromic compound according to the disclosure.

FIG. 6 illustrates an embodiment of a product for coloring a substrate according to the disclosure.

FIG. 7 illustrates another embodiment of a product for coloring a substrate according to the disclosure.

FIG. 8 illustrates another embodiment of a product for coloring a substrate according to the disclosure.

FIG. 9 illustrates another embodiment of a product for coloring a substrate according to the disclosure.

FIG. 10 illustrates another embodiment of a product for coloring a substrate according to the disclosure.

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

### DETAILED DESCRIPTION OF THE INVENTION

This disclosure relates to photochromic compounds that are free from C—F bonds that are typical of many conventional photochromic compounds, but which are a type of polyfluoroalkyl substance (PFAS). The disclosed photochromic compounds include a fluorine-free bridge, heterocycle, and aromatic structures that provide reversible color switching upon activation by electromagnetic radiation (“EMR”). Selected functional groups for the aromatic structures allow control of several properties of the photochromic compounds, including the specific color resulting from activation, the speed with which the compound can be activated, the length of time that the activated color can persist in ambient deactivating light, and chemical compatibility/solubility of the photochromic compounds with common solvents, carriers, and other ingredients common for cosmetic compositions suitable for topical use. One or more of the photochromic compounds can be incorporated into a coloring composition for reversibly (or in some cases, irreversibly) coloring a substrate, in particular including human hair, but also including other substrates such as skin, textiles, and fabrics. Coloring compositions including multiple different photochromic compounds can be used to provide a tunable activated color based on selective activation or deactivation of individual photochromic compounds. Also disclosed are methods for coloring a hair or other substrate using the photochromic compound. Further disclosed are products incorporating the photochromic compound along with a light source for activating and/or deactivating the photochromic compound.

### Definitions

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges not explicitly delineated within these ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

“About” modifies a particular value by referring to a range of plus or minus 20% or less of the stated value (e.g., plus or minus 15% or less, 10% or less, or even 5% or less).

“Activate” and variations thereof, when referring to a photochromic dye or a composition comprising a photochromic dye, means causing the photochromic dye and/or composition to change color by exposing it to electromagnetic radiation (“EMR”) of a particular wavelength or changing the temperature of the dye.

“Color” refers to visibly perceived light of a particular wavelength, generally between 400 nm and 700 nm. Color may be characterized in terms of Hue, Saturation, and Lightness, and can be measured using conventional methods, for example, with a spectrophotometer or colorimeter.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the U.S. Food and Drug Administration.

“Deactivate” and variations thereof, when referring to a photochromic dye or a composition comprising a photochromic dye, means causing the photochromic dye and/or composition to revert back to substantially the same color it was prior to activation by exposing the dye to EMR of a particular wavelength, removing activating EMR or changing the temperature of the dye. “Different colors” and variations thereof mean colors that have a different Hue, Saturation and/or Lightness value relative to one another. In some aspects, different colors can be characterized by having a ΔE of greater than 1, relative to one another, according to the Color Measurement method.

“Fluence” means the time-integrated flux of EMR (i.e., activating EMR and deactivating EMR) on an irradiated surface. Fluence can be readily determined by those skilled in the art.

“Suitable for application to human hair” means that the composition or components thereof, are acceptable for use in contact with human hair and the scalp and skin without undue toxicity, incompatibility, instability, and the like.

“Substantially free of” means a composition or ingredient contains less than 3% (e.g., less than 2%, 1% or even less than 0.5%) of a subject material, by weight of the composition or ingredient. “Free of” means 0% of a subject material is present in the composition or ingredient.

“Tunable” means that the hair color change provided by the photochromic hair dye compositions herein can be adjusted to a desired color. The hair color change may be tuned, for example, by changing: the activating/deactivating time, the strength of the EMR source, the concentration or ratio of photochromic dyes and/or the activating/deactivating technique (e.g., how close the EMR source is to the hair or selectively exposing target portions of hair to the EMR source).

“Visible color change” and variations thereof mean a color change that results in a ΔE of more than 1 (e.g., 2, 3, 5, 10, 20 or more). ΔE can be determined according to the Color Measurement method described in more detail below.

### Photochromic Compound

Photochromic compounds (or dyes) are generally recognized as compounds that undergo a reversible color change when exposed to EMR of a particular wavelength and fluence (“activating EMR”). Photochromic compounds are classified into two types, p-type and t-type. When a photochromic compound is exposed to an activating and/or deactivating EMR it undergoes a molecular transformation between isomers of the photochromic compound molecule, for example between a base (unactivated) state for the compound and an activated analog of the compound in a different color state as described in more detail below. In some case, prolonged exposure to activating EMR can further transform the photochromic compound to a dyotropic rearrangement of the compound (e.g., from the activated analog), which is a generally permanent, stable, and/or irreversible transformation of the compound to a third color state (e.g., same or different from that of the activated analog), as described in more detail below.

P-type photochromic compounds are generally thermally irreversible, but photochemically reversible. In other words, a p-type photochromic compound will change color when irradiated with activating EMR but will generally not revert back to its original color when the activating EMR is removed or when the temperature of the dye changes. The p-type photochromic compound suitable for use herein can be activated by an activating EMR having a wavelength of 250 nm to 395 nm (e.g., 260 nm to 380 nm or 270 nm to 370 nm) and a fluence of 1 mJ/cm² to 50 J/cm². A p-type photochromic dye is deactivated by exposing the dye to deactivating EMR, which has a different wavelength from the activating EMR. In some embodiments, the deactivating EMR has a wavelength of 400 to 1400 nm (e.g., 400 to 700 nm; 425 nm to 650 nm) and a fluence of 1 mJ/cm² to 50 J/cm². The photochromic compounds suitable for use herein may have an activation and/or deactivation time of less than 1 second up to 10 minutes (e.g., 0.001 seconds to 5 minutes, 0.01 second to 3 minutes, 0.1 second to 2 minutes seconds, 0.5 second to 90 seconds, or even 10 seconds to 60 seconds). Relatively shorter activation and/or deactivation times may be more convenient to a user, and therefore preferred.

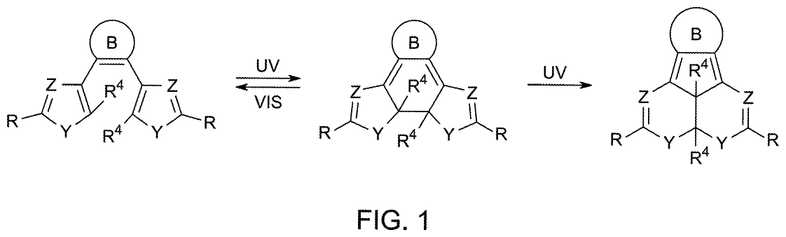

Photochromic compounds according to the disclosure include p-type fluorine-free photochromic compounds that can exist in one, two, or three related states or forms: a fluorine-free first compound, a fluorine-free second compound that is an activated analog of the fluorine-free first compound, and a fluorine-free third compound that is a dyotropic rearrangement of the fluorine-free first compound (or of the fluorine-free second compound). The fluorine-free first compound has a cyclic bridge (e.g., having 4, 5, or 6 ring atoms) with one ethylenically unsaturated bond (C═C). The fluorine-free first compound further includes first and second substituents (same or different) bonded to opposing carbon atoms in the ethylenically unsaturated bond. The first substituent includes a first unsaturated heterocycle (e.g., thiophene or otherwise) having a first aromatic group or a first halogen (e.g., Cl, F) bonded to the first unsaturated heterocycle, for example at a carbon atom of the heterocycle. The second substituent includes a second unsaturated heterocycle (e.g., thiophene or otherwise) having a second aromatic group bonded to the second unsaturated heterocycle, for example at a carbon atom of the heterocycle. The first and second aromatic groups can be substituted with one or more linear or branched alkyl or alkylene oxide groups, one or more hydroxyl groups, one or more amino or ammonium groups, and combinations thereof, for example including 1, 2, 3, 4, or 5 non-hydrogen substituents on the aromatic groups, each with one or more of the various linking groups described below between the aromatic ring and the aromatic substituent(s). The fluorine-free first compound can include the Formula (I) compound described below. The activated analog structure and the dyotropic rearrangement structure for the fluorine-free second and third compounds, respectively, are illustrated below for the representative case of the Formula (I) base/unactivated structure (FIG. 1). The first and second fluorine-free compounds are thermally irreversible, but photochemically reversible between each other with application of activating or deactivating EMR. The transformation of the second fluorine-free compound to the third fluorine-free compound is thermally and photochemically irreversible.

A photochromic compound according to the disclosure can be represented by Formula (I), which represents the unactivated or base state of the photochromic compound. As described above, the photochromic compound can exist in multiple related states: the unactivated or base state of Formula (I), an activated analog of Formula (I) (e.g., reversibly activated by UV EMR), and a dyotropic rearrangement Formula (I) or its activated analog (e.g., irreversibly activated by further UV EMR). Formula (I) is illustrated below as having a bridge B and two substituents A¹ and A² at the ethylenic bond of the bridge, where A¹ and A² can have the aromatic-substituted heterocycle or the halogen-substituted heterocycle structures of Formulas (Ia) and (Ib), respectively:

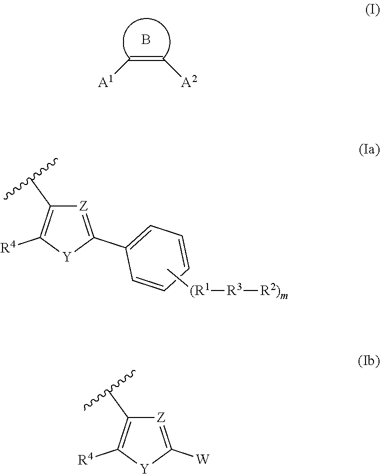

In Formula (I), the bridge or ring B has the formula

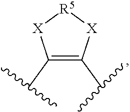

X is CH₂ and R⁵ is a bond (i.e., absent) or unsubstituted or substituted C₁₋₂ alkylene (e.g., 1 or 2 carbon atoms); or X is carbonyl and R⁵ is O, NH, or NR⁸. R⁸ is independently selected from H, C₁₋₆ alkyl (e.g., 1, 2, 3, 4, 5, or 6 carbon atoms), and aryl. The bridge can have 4, 5, or 6 ring atoms. Illustrative structures for the ring B are

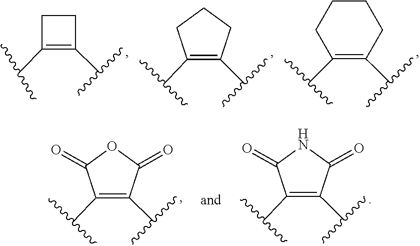

In Formula (I), A¹ and A² can independently be represented by Formula (Ia) and or Formula (Ib), with the proviso that at least one of A¹ and A² is a substituent having Formula (Ia). In some embodiments, A¹ is identical to A², and both are represented by Formula (Ia).

In Formulas (Ia) and (Ib), each Y is independently selected from S, O, and NR⁶; and each Z is independently selected from N and CR⁷. R⁶ and R⁷ are independently selected from H, C₁₋₆ alkyl (e.g., 1, 2, 3, 4, 5, or 6 carbon atoms), and aryl. In embodiments, Y is S and Z is CH to represent a thiophene heterocycle. Each R⁴ is independently selected from C₁₋₈alkyl (e.g., 1, 2, 3, 4, 5, 6, 7, or 8 carbon atoms), C₄₋₈cycloalkyl (e.g., 4, 5, 6, 7, or 8 carbon atoms), C₁₋₁₂alkoxy (e.g., 1, 2, 3, 4, 5, 6, 7, 8, 9, 10, 11, or 12 carbon atoms), C≡CR⁷, and cyano.

In Formula (Ia), each R¹ is independently selected from O, S, C═C, C≡C, OC(═O), C(═O)O, C(═O)S, SC(═O), C(═O)NH, NHC(═O), C(═S)NH, NHC(═S), NHC(═O)NH, and NHC(═S)NH. For each of the foregoing groups, the left side of the group can be that which is bonded to the aromatic ring, and the right side of the group can be that which is bonded to R³-R². For example, when R¹ is C(═O)O, then the corresponding structure in Formula (Ia) can be represented by [aromatic ring]-C(═O)O—R³-R². Each R² is independently selected from H, C₁₋₆alkyl (e.g., 1, 2, 3, 4, 5, or 6 carbon atoms), N(R⁸)₂, N⁺H(R⁸)₂, and any of the foregoing linked to R³ via an additional R¹ group. R⁸ is independently selected from H, C₁₋₆ alkyl (e.g., 1, 2, 3, 4, 5, or 6 carbon atoms), and aryl. Each R³ is independently a bond (i.e., absent such that R¹ and R² are directly bonded), linear or branched C₁₋₂₀alkylene (e.g., 1, 2, 3, 4, 5, 6, 7, 8, 9, 10, 11, 12, 13, 14, 15, 16, 17, 18, 19, or 20 carbon atoms), or linear or branched (OCH₂CH₂)ₙ, where n is an integer in the range of 1 to 20 (e.g., 1, 2, 3, 4, 5, 6, 7, 8, 9, 10, 11, 12, 13, 14, 15, 16, 17, 18, 19, or 20 ethylene oxide units). Each m is independently 1 to 5, for example 1, 2, 3, 4, or 5 of the same or different —R¹-R³-R² substituents attached to the aromatic or phenyl (Ph) group at any combination of ortho, meta, and para locations relative to the bond to the heterocycle.

Suitably, the —R¹-R³-R² substituent is bonded to the aryl ring at the para position relative to the bond to the 5-member heterocycle. In other embodiments, the R¹-R³-R² substituent can be bonded at ortho or meta positions relative to the bond to the 5-member heterocycle.

Formula (Ia) is illustrated with a single —R¹-R³-R² substituent. More generally, the aromatic ring in Formula (Ia) can be substituted with up to five substituents (same or different), for example being represented by [R¹-R³-R²]n, where n=1, 2, 3, 4, or 5 and R¹, R³, and R² can be independently selected from the options above for each n.

In Formula (Ib), W is a halogen, suitably Cl, Br, or I.

The photochromic compound can include any combination or distribution of its different forms, including its unactivated base form (e.g., Formula (I) compound), activated analog form (e.g., activated analog of Formula (I)), and dyotropic rearrangement form (e.g., dyotropic rearrangement of Formula (I)). Each form can independently be absent, substantially absent, or present in any amount relative to all forms combined, for example with each form independently being present in an amount of at least and/or up to 0, 1, 2, 5, 10, 20, 25, 30, 40, 50, 60, 70, 75, 80, 90, 95, 98, 99, or 100 mol. % and ranges therebetween relative to all three forms. Alternatively or additionally, any two forms can be present in relative molar ratios of 1:99 to 99:1, or 5:95 to 95:5, or 10:90 to 90:10, or 25:75 to 75:25.

Photochromic compounds and corresponding coloring compositions of the disclosure can be activated by exposure to an activating EMR. Activating a photochromic compound of the disclosure can convert some, substantially all, or all of the compound to an activated state (e.g., second or third fluorine-free compound). The activating EMR can include EMR of one or more wavelengths that are absorbed by the compound. The activating EMR can include EMR having wavelengths in a range of 100 nm to 400 nm, or 250 nm to 395 nm, or 250 nm to 350 nm, or 260 nm to 340 nm, or 270 nm to 330 nm, or 280 nm to 320 nm. The wavelength of the activating EMR can be about 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 395 nm, for example, or within a range formed by any such values as endpoints. The activating EMR can also include EMR having wavelengths that are not absorbed by the compound. The source of the activating EMIR is not particularly limited, for example including a lamp, LED, laser, etc. emitting light at a specific wavelength or range of wavelengths.

Similarly, activated analogs of photochromic compounds of the disclosure (e.g., second fluorine-free compound) can be deactivated by exposure to a deactivating EMR. Deactivating the activated analog of a compound of the disclosure can convert some, substantially all, or all of the activated analog to the compound's structure prior to activation (e.g., first fluorine-free compound). The deactivating EMR can include EMR of one or more wavelengths that are absorbed by the activated analog of the compound of the disclosure. The deactivating EMR can comprise EMR having wavelengths in a range of 400 nm to 1400 nm, or 400 nm to 1200 nm, or 400 nm to 1000 nm, or 400 nm to 900 nm, or 400 nm to 800 nm, or 400 nm, to 700 nm, or 450 nm to 650 nm, or 500 nm to 600 nm. The wavelength of the deactivating EMR can be about 400 nm, 425 nm, 450 nm, 475 nm, 500 nm, 525 nm, 550 nm, 575 nm, 600 nm, 625 nm, 650 nm, 675 nm, 700 nm, 800 nm, 900 nm, 1000 nm, 1100 nm, 1200 nm, 1300 nm, or 1400 nm, for example, or within a range formed by any such values as endpoints. The deactivating EMR can include EMR having one or more wavelengths that are absorbed by the activated analog. The deactivating EMR can also include EMR having wavelengths that are not absorbed by the activated analog. The source of the deactivating EMR is not particularly limited, for example including a lamp, LED, laser, etc. emitting light at a specific wavelength or range of wavelengths. For instance, the source of the deactivating EMR can be a white light source.

Compounds of the disclosure can be characterized by a 50% activation time, denoted herein as t_{1/2,a}. A 50% activation time of a sample including a compound of the disclosure is the time in which the absorbance of the sample increases to 50% of the final or asymptotic absorbance of the sample after prolonged exposure to activating EMR, for example after conversion of substantially all of the compound to its activated analog upon exposure to activating EMR, or after conversion of a sufficient portion of the compound to its activated analog to achieve it final or most intense color state. The 50% activation time is a measure of the rate of formation of the activated analog and is accordingly a measure of the rate of color change the compound undergoes upon exposure to activating EMR. Absorbance for determining a 50% activation time can be measured at any wavelength at which the activated analog absorbs, such as the wavelength of peak absorbance of the activated analog. The 50% activation time is preferably on the order of seconds. The 50% activation time can be 30 seconds or less, or in a range of 1 to 600 seconds, for example at least and/or up to 1, 2, 3, 5, 7, 10, 15, 20, 30, 40, 50, 60, 90, 120, 180, 240, 300, 360, 480, or 600 seconds and ranges therebetween.

Compounds of the disclosure can also be characterized by a 50% deactivation time, denoted herein as t_{1/2,d}. A 50% deactivation time of a sample including the activated analog of a compound of the disclosure is the time in which the absorbance of an initially fully activated sample decreases to 50% of its initial absorbance upon exposure to deactivating EMR. The 50% deactivation time is a measure of the rate of disappearance of the activated analog and is accordingly a measure of the rate of color change the activated analog undergoes upon exposure to deactivating EMR. Absorbance for determining a 50% deactivation time can be measured at any wavelength at which the activated analog absorbs, such as the wavelength of peak absorbance of the activated analog. The 50% deactivation time is preferably on the order of minutes. The 50% deactivation time can be 30 minutes or more, or in a range of 2 to 600 minutes, for example at least and/or up to 2, 3, 5, 7, 10, 15, 20, 30, 40, 50, 60, 90, 120, 180, 240, 300, 360, 480, or 600 minutes and ranges therebetween.

The photochromic compound of the disclosure can be non-phototoxic at concentrations up to 100 μg/mL as measured by the OECD 432 Assay (NRU uptake phototoxicity test). Alternatively or additionally, the photochromic compound of the disclosure can be non-photoreactive at concentrations up to 100 μg/mL as measured by the OECD 495 Assay (ROS photoreactivity test). Other concentration thresholds can be used for either test, for example 1, 2, 5, 10, 20, 50, 100, 200, 500, or 1000 μg/mL.

FIG. 1 illustrates a photochromic compound according to the disclosure in an unactivated base state (left), an activated analog state (middle), and a dyotropic rearrangement state (right). The unactivated base state on the left illustrates Formula (I) in which R represents the aromatic or halogen substituent on the heterocycle (e.g., according to Formula (Ia) or (Ib), respectively). With relatively short activation times/energies in the UV range, the Formula (I) compound can transform to its activated analog state shown in the middle of FIG. 1. The activated analog state can reversibly transform back to the unactivated base state when subjected to deactivating visible (or infrared) light. In other cases, at prolonged activation times/energies in the UV range, the activated analog state can irreversibly further transform into its dyotropic rearrangement state shown on the right of FIG. 1.

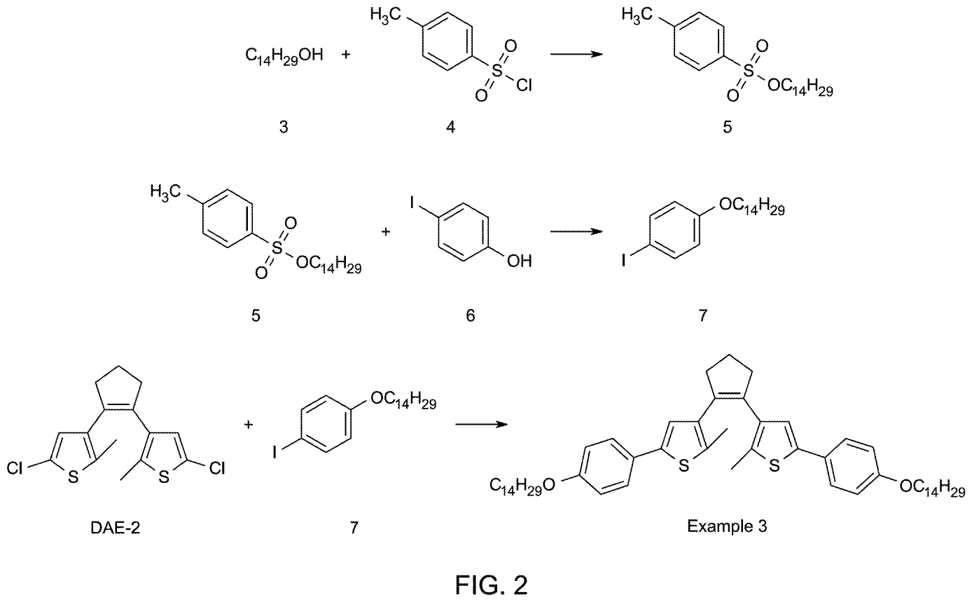

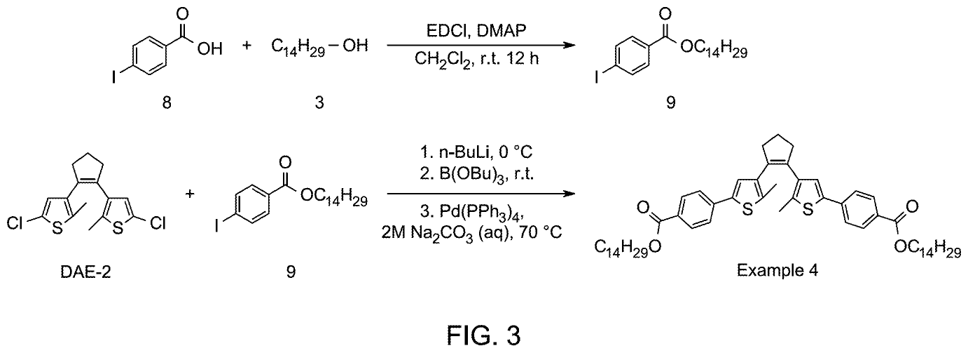

Compounds of Formula (I) can be prepared by suitable synthetic techniques generally known in the art, for example, by reacting an aryl halide with a diarylethene compound having one or more halogenated heterocycle substituents using Suzuki coupling conditions. Examples of this coupling step are shown in FIGS. 2 and 3, in the last step of syntheses of Examples 3 and 4, respectively. As shown, substituents on the aryl halide (for instance, (7) and (9) with iodine substituents in FIGS. 2 and 3, respectively) correspond to the R¹-R³-R² group in the resulting compound of Formula (I). The Suzuki coupling step accordingly provides a versatile means of installing various functional groups on compounds of Formula (I). The halogen atoms used in the aryl group suitably can be iodide and bromide, however, an aryl iodide is preferred for enhancing the yield in the Suzuki reaction.

Substituted aryl halides for use in the preparation of compounds of Formula (I) can be prepared by any suitable means.

As is further shown in FIGS. 2 and 3, the diarylethene compound having one or more halogenated heterocycle substituents provides the ring B (i.e., cyclic bridge) and heterocycle substituents on the resulting compound of Formula (I). One suitable diarylethene compound for preparing compounds of Formula (I) is 1,2-bis(5-chloro-2-methylthiophen-3-yl)cyclopentene (DAE-2). As will be evident to the skilled person, other diarylethene compounds including a different cyclic bridge (e.g., a cyclobutene, cyclohexene, maleic anhydride, or maleimide group) and/or different heterocycles (e.g., thiazole) can also be suitable for preparing compounds of Formula (I).

The diarylethene compound having halogenated heterocycle substituents can be prepared, for example, by reacting a diacid halide and a halogenated heterocycle compound via Friedel-Crafts acylation. The diacid halide can be a linear α,ω-diacid chloride, such as succinyl chloride, glutaryl chloride, or adipoyl chloride. The resulting product, having the dione groups and two terminal heterocycle substituents, can form the cyclic diarylethene bridge when reacted with TiCl₄ and Zn via a McMurry reaction.

### Coloring Composition

As described in more detail below, the coloring composition is suitable for application to any of a variety of substrates or surfaces to provide a convenient way for a user to change, adjust, or tune the color of the substrate. The substrate is not particularly limited and can include, for example a keratinous surface such as hair or skin on any portion of a user's body, a textile or fabric substrate such as a clothing item, etc. In some embodiments, the coloring composition is a hair coloring composition that can be used to tune the color of a user's hair between two or more visibly different colors, for example the original hair color, a first new color, and a second new color.

The coloring composition can include one or more photochromic compounds according to the disclosure, one or more carrier components such as a solvent, and one or more optional ingredients such as those suitable for hair coloring compositions (e.g., hair dyes, shampoos, conditioners, hair styling products), skin care compositions, and other cosmetic compositions. The photochromic compound may each be present in the coloring composition at 0.01% to 25% (e.g., 0.05% to 20%, 0.1% to 10%, 0.5% to 5%, or even 1% to 3%), for example at least and/or up to 0.01, 0.05, 0.1, 0.2, 0.5, 1, 2, 3, 5, 10, 15, 20, or 25% and ranges therebetween for an individual photochromic compound (e.g., including its unactivated base, activated analog, and dyotropic rearrangement forms combined) and/or for all photochromic compounds combined. The coloring composition can include a suitable solvent to ensure that the photochromic compound is suitably dispersed in the carrier. The solvent may be present at 1% to 99.99% (e.g., 2% to 90%, 3% to 80%, 4% to 70%, or even 5% to 50%), for example at least and/or up to 1, 2, 3, 4, 5, 10, 20, 30, 40, 50, 60, 70, 80, 90, or 99.99% and ranges therebetween for an individual solvent and/or for all solvents combined. Other carrier components (e.g., other than the solvent) can be present in amounts of 1% to 99.99% and analogous sub-ranges. The pH of the coloring composition may be between 2 and 10 (e.g., 2, 3, 4, 5, 6, 7, 8, 9, 10, or ranges therebetween).

The coloring compositions herein may be provided in various product forms such as solutions, suspensions, shampoos, conditioners, lotions, creams, gels, sticks, sprays, aerosols, ointments, solid bars, pastes, foams, mousses, hydrogels, and the like. The composition form may follow from the particular dermatologically acceptable carrier chosen.

The coloring composition can include the photochromic compound in any of its forms, including its unactivated base form (e.g., first fluorine-free compound or Formula (I) compound), activated analog form (e.g., second fluorine-free compound or activated analog of Formula (I)), and dyotropic rearrangement form (e.g., third fluorine-free compound or dyotropic rearrangement of Formula (I)). Each form can independently be absent, substantially absent, or present in any amount relative to all forms combined, for example with each form independently being present in an amount of at least and/or up to 0, 1, 2, 5, 10, 20, 25, 30, 40, 50, 60, 70, 75, 80, 90, 95, 98, 99, or 100 mol. % and ranges therebetween relative to all three forms. Alternatively or additionally, any two forms can be present in relative molar ratios of 1:99 to 99:1, or 5:95 to 95:5, or 10:90 to 90:10, or 25:75 to 75:25 (e.g., for first:second forms, second:third forms, first:third forms). In some embodiments, the most intense or ultimate color change for the photochromic compound can be obtained when about 50-90% of the photochromic compound is in an activated state and about 10-50% of the photochromic compound is in the unactivated state (e.g., a ratio of activated:unactivated molecules of 1:1, 2:1, 3:1, 4:1, 5:1, 6:1, 7:1, 8:1, 9:1, or ranges therebetween). The foregoing distributions and ranges for the different photochromic compound forms can apply to the photochromic compound or coloring composition either before or after activation and/or deactivation (e.g., with different distributions before and after).

In embodiments, the coloring composition includes the activated analog form and/or the dyotropic rearrangement form of the photochromic compound, for example as initially packaged or sold. In embodiments, the coloring composition includes the unactivated form of the photochromic compound, for example as initially packaged or sold. More generally, the initial coloring composition as provided to a user can include a blend between un-activated, activated, and rearrangement forms, such as 1-99 mol. % of each relative to total components. For example, the initial coloring composition can contain essentially all activated and/or rearrangement forms of the photochromic compound, such as at least 95, 98, or 99 mol. % of the activated and/or rearrangement forms. Alternatively, the initial coloring composition can contain essentially all unactivated forms of the photochromic compound, such as at least 95, 98, or 99 mol. % of the unactivated forms.

In some aspects, the coloring compositions include a plurality of different photochromic compounds, for example two, three, four, or more different photochromic compounds. For example, the coloring composition may include two or more p-type photochromic compounds, a p-type photochromic compound and a t-type photochromic compound, or a combination of a photochromic compound and a non-photochromic hair dye. At least one of the photochromic compounds corresponds to one or more forms of the disclosed photochromic compound, for example the first, second, and/or third fluorine-free compounds and/or the Formula (I) compounds, analogs, and rearrangements. In some embodiments, all of the different photochromic compounds correspond to one or more forms of the disclosed photochromic compound. A given photochromic compound (or “single” compound) with particular color properties includes any or all of the unactivated base forms, the activated analog forms, and the dyotropic rearrangement forms.

In some embodiments, the coloring composition can include one or more conventional photochromic compounds (e.g., non-fluorinated conventional photochromic compounds). Suitable conventional p-type photochromic compounds include those belonging to the diarylethene (“DAE”) family and/or fulgide family. Some non-limiting examples of diarylethene and fulgide photochromic dyes are described in U.S. Pat. No. 6,123,952 and US20120017929. Some particularly suitable examples of p-type photochromic compounds for use herein include DAE-0001 (cyan), DAE-0012 (magenta), DAE-0068 (yellow) from Yamada Chemical Company. Examples of p-type photochromic compounds include (1) 4,4′-(perfluorocyclopent-1-ene-1,2-diyl)bis(5-methyl-2-phenylthiazole), which changes from clear to magenta when exposed to a suitable activation energy; (2) 5,5′-(perfluorocyclopent-1-ene-1,2-diyl)bis(2,4-dimethylthiophene), which changes from clear to yellow when exposed to a suitable activation energy; and (3) 3,3′-(perfluorocyclopent-1-ene-1,2-diyl)bis(2-methyl-5-phenylthiophene), which changes from clear to cyan when exposed to a suitable activating energy. Other examples of p-type photochromic compounds include (1) (E)-alpha-2,5-Dimethyl-3-furylethylidene(isopropylidene)succinic anhydride (ABERCHROME 540; color change from pale yellow to red); (2) (E)-3-(Adamantan-2-ylidene)-4-[1-(2,5-dimethyl-3-furyl)ethylidene]dihydro-2,5-furandione (ABERCHROME 670; color change from pale yellow to intense red); (3) p-methylacetophenylisopropylidenesuccinic anhydride (showing light magenta after irradiation); (4) p-chloroacetophenylisopropylidenesuccinic anhydride (showing brownish orange after irradiation); (5) p-bromoacetophenylisopropylidenesuccinic anhydride (showing orange after irradiation).

In some embodiments, the coloring composition may include one or more t-type photochromic dyes. T-type photochromic dyes are thermally reversible, which generally means they will revert back to their original color once the activating EMR is removed. T-type photochromic dyes include azobenzene and spiropyran compounds. Some non-limiting examples of T-type photochromic dyes can be found in U.S. Pat. Nos. 5,581,090, 5,730,961, and FR1604929.

In embodiments, the coloring composition can include a first photochromic compound having a first color in activated (or rearrangement) form, a second photochromic compound having a second color in activated (or rearrangement) form, optionally a third photochromic compound having a third color in activated (or rearrangement) form, optionally a fourth photochromic compound having a fourth color in activated (or rearrangement) form, and so on, for example including fifth, sixth, seventh, or eighth (or more) additional photochromic compounds and corresponding colors. The first, second, third, fourth, etc. colors can be different colors or otherwise distinct from each other. For example, the different colors of the photochromic compounds can be selected from cyan (C), magenta (M), yellow (Y), and/or black (K) activated colors or red (R), green (G), and/or blue (B) activated colors following the color theory of mixing to provide tunable composite colors for the activated composition.

When the coloring composition includes the plurality of different photochromic compounds, each photochromic compound can be selected such that at least one of its characteristic activation wavelength (λₐ) and its characteristic deactivation wavelength (λ_{d}) is different by at least 60 nm relative to a characteristic activation wavelength or a characteristic deactivation wavelength, respectively, of a different photochromic compound in the coloring composition (or relative to all other photochromic compounds in the composition). The characteristic wavelengths can represent peaks in absorption spectra for the photochromic compound at which the greatest degree of activation or deactivation, respectively, is observed. While it is preferable to have characteristic wavelengths different by at least 60 nm to promote selective activation or deactivation, more generally differences in wavelength of at least and/or up to 20, 30, 40, 50, 60, 70, 80, 100, 120, 160, 200 nm, or ranges therebetween are possible. Photochromic compounds having distinct activation/deactivation wavelengths facilitate color tuning by selectively activating/deactivating a single color in the mixture. In other cases, different photochromic compounds might have similar activation/deactivation wavelengths, but they might absorb EMR at different rates, require different amounts of energy for a given color change, etc., so color tuning via selective activation/deactivation can still be possible even with overlapping/very close characteristic wavelengths.

When the coloring composition includes the plurality of different photochromic compounds, each photochromic compound can be selected such that each photochromic compound has a 50% ambient deactivation time (t_{1/2,d}) that is within 50% (e.g., within +/−5, 10, 20, 30, 40, or 50%) of the t_{1/2,d} values for the other photochromic compound(s). This feature reflects a mixture of photochromic compounds that individually fade at similar rates in ambient light so that the initially activated mixture retains substantially the same blend of different colors even as their intensities fade. The +/−percent thresholds can alternatively be compared to an average t_{1/2,d} for photochromic compounds in the mixture.

In some embodiments, the photochromic compounds are included in the coloring composition at a ratio of first photochromic compound to second photochromic compound of 1:50 to 50:1 (e.g., 1:30, 1:20, 1:10, 1:5, 1:3, 1:2, 1:1, 2:1, 3:1, 5:1, 10:1, 20:1, or 30:1), depending on the desired hair color change. Of course, one or more additional photochromic compounds may also be included at a suitable ratio to the first and/or second photochromic compound. It is to be appreciated that the ratio of the photochromic compounds in the composition can be important for providing the desired color change, for example a hair color change to users who have different hair colors. For example, to provide the same or similar color of hair to a user with blond hair and a user with black hair, it may be necessary for the ratio of photochromic compounds in the coloring composition to be different.

Hair color can be classified on a scale of 1 to 10 based on well-known hair color charts such as the one in Table 1 below.

TABLE 1
Hair Color Chart

| 1 | 2 | 3 | 4 | 5 | 6 | 7 | 8 | 9 | 10 |
|---|---|---|---|---|---|---|---|---|---|
| Lightest | Medium | Dark | Lightest | Medium- | Medium | Dark | Darkest | Black | Darkest |
| Blonde | Blonde | Blonde | Brown | light | Brown | Brown | Brown | | Black |

The hair color values shown in the chart can readily be correlated to red, green, and blue color values based on the RGB color models, for example, as described in US 2021/0308020. Additionally or alternatively, the hair color values can be correlated to other known color values/models such as: cyan, magenta, yellow, black (CMYK) color values; L*a*b* color values, as defined by the International Commission on Illumination (CIE Lab); or Hue, Saturation, and Lightness (HSL) color values. By tailoring the type and amount of photochromic compound in the composition based on the color interactions of known color models, it is possible to provide a user with a more predictable hair color and a wider range of tunable color options.

Providing temporary color effects to medium or darker colored hair is known to be problematic. Conventional hair coloring systems may resort to pre-treating darker colored hair to lighten the color of the hair, of the hair, sometimes referred to as bleaching and then applying a desired final color. However, bleaching is known to be harmful to hair. By tailoring the combination of photochromic compounds to the user's hair color, the coloring compositions described herein can provide a desired visible hair color change without the need to lighten or otherwise pretreat the hair.

The coloring compositions herein provide an excellent aesthetic appearance to hair. In particular, the hair dye compositions herein can provide excellent Hue, Saturation, Lightness, and/or Gloss to the hair. “Hue” refers to the color obtained and corresponds to value ranging from 0° to 360° (e.g., 0° (red), 30° (orange), 60° (yellow), 90°, 120° (green), 150°, 180° (cyan), 210°, 240° (blue), 270° (violet), 300° (magenta), or 330°). “Saturation” is the intensity of the color and has a value ranging from 0% (gray) to 100% (pure color). “Lightness” is the amount of white or black mixed with the color to provide various tints and shades. The Lightness value can be 0% (black) to 100% (white). Gloss is the amount of light reflecting from a surface which gives the effect of shine and can range from 1 to 100, where a lower value indicates lower gloss/higher matte. HSL values can be measured using a spectrophotometer according to the Color Measurement method described in more detail below. Gloss can be measured concurrently with HSL, for example, by setting the SCI/SCE mode on a suitable spectrophotometer to include a specular component (SCI mode) whereby reflected light from a surface is quantified as the Gloss value. Additionally or alternatively, L*a*b* colors values can be measured with a suitable spectrophotometer and converted to HSL color values.

In some aspects, the coloring composition herein may be provided to a user in a package that enables the user to activate and/or deactivate the photochromic compounds in the package, for example, by irradiating the coloring composition with EMR through a transparent portion of the package. The coloring composition, when activated and/or deactivated in its package, may have the same or different color properties from what it has when applied to hair and then activated and/or deactivated. In some instances, the coloring composition may be exposed to EMR in its package to partially activate and/or deactivate it, and then applied to the hair and exposed to additional EMR to further activate and/or deactivate the coloring composition.

The coloring compositions herein enable a user to conveniently change the visible color of their hair to a variety of different colors with a single hair dye composition. The hair dye composition may be tailored to deliver a visible color change based on the fluence of the activating EMR, the wavelength of the activating EMR. the fluence of the deactivating EMR, the wavelength of the deactivating EMR, the activation time of the photochromic compound, and/or the deactivation time of the photochromic compound. In some aspects, the coloring compositions herein may provide long lasting color change (e.g., more than 2 hours, 4 hours, 8 hours, 12 hours, 18 hours, or even 24 hours or more) upon activation and/or deactivation.

The color of the photochromic compounds in the hair dye composition, when activated, are not particularly limited and may be any color, as desired. In some instances, it may be desirable for the activated dye color to be selected from cyan, magenta, yellow, and combinations thereof to take advantage of CMYK printing principles to provide a wide range of different colors. Cyan may be characterized as having an HSL value of 300°, 100% and 50%. Magenta may be characterized as having an HSL value of 180°, 100% and 50%. Yellow may be characterized as having an HSL value of 60°, 100% and 50%. In some instances, a color that has a Hue value within 3°, a Saturation value within 10%, and a Lightness value within 10% of the target value for a particular color may be considered that color. For example, a color with an HSL value of 178°, 95%, 45% may be considered Cyan.

### Carrier

The coloring composition herein can include 1%-95% of a cosmetically acceptable carrier. The carrier will typically be liquid, but embodiments in which the carrier is solid or semi-solid (e.g., gel) are also contemplated herein. The carrier may include polar and/or non-polar materials and may be in the form of a single phase (e.g., solution or dispersion) or multiple phases (e.g., an oil-in-water or water-in-oil emulsion). In some embodiments, the carrier includes a solvent in which the photochromic compounds are soluble. The solvent may make up some or all of the carrier (e.g., 5% to 100%). In some embodiments, the photochromic compound has a solubility of 1 mg/L to 500 g/L in the solvent, for example at least and/or up to 0.001, 0.01, 0.1, 1, 2, 5, 10, 20, 50, 100, 200, 300, 400, or 500 g/L and ranges therebetween in the solvent at about 20° C., 25° C., 40° C., or 50° C. Some non-limiting examples of solvents that may be suitable for dissolving DAE photochromic dyes include emulsifiers (e.g., TWEEN® 20, TWEEN® 40, TWEEN® 60, and TWEEN® 80 brand polysorbate and SPAN® 20, SPAN® 40, SPAN® 60 and SPAN® 80 brand sorbitan laurate), fatty acids (e.g., steric acid, oleic acid, and palmitic acid), fatty alcohols (e.g., cetyl alcohol, lauryl alcohol, stearyl alcohol, oleyl alcohol, and octyldodecanol), fatty esters (e.g., caprylic/capric triglyceride, isopropyl palmitate, and isopropyl myristate), silicones (e.g., cyclopentasiloxane, cyclohexasiloxane, dimethicone, and methicone) and botanical oils (e.g., wheatgerm oil, beauty-leaf oil, sesame oil, macadamia oil, grapeseed oil, rapeseed oil, coconut oil, groundnut oil, palm oil, castor oil, jojoba oil, olive oil or cereal germ oil, cedarleaf oil, turpentine oil, and eucalyptus oil) and combinations of these. Botanical oils may be particularly suitable because they can be naturally and/or sustainably sourced and are generally perceived by consumers as being better for their body.

### Optional Ingredients

The coloring compositions herein may include other optional ingredients commonly found in hair coloring compositions and/or other cosmetic hair and/or skin care compositions. These additional ingredients may be present at 0.5% to 95%. Some non-limiting examples of optional ingredients can be found in U.S. Pat. No. 7,056,351 and US2002/0053110 and may include non-photochromic hair dyes, stabilizing agents (e.g., UV stabilizing agents), binding agents, deposition aids, organic acids, surfactants, hair or skin conditioning agents, anti-dandruff actives, fungicides, alkyl ethoxylate hydrocarbons, silicone compounds, cationic polymers, proteins, amino acids, preservatives, moisturizing agents, viscosity modifiers, emulsifiers, pH modifiers, buffering agents, chelants, and combinations of these.

### Method of Making Composition

In some embodiments, the coloring compositions may be made using conventional methods of making such compositions. In some embodiments, it may be desirable to combine the photochromic compound and a solvent into a premix, which is then added to the carrier and/or other ingredients in the coloring composition.

### Method of Using Composition

The coloring compositions herein can be applied to a target portion of a substrate where a color change is desired. The substrate is not particularly limited, but it suitably can be a keratinous substrate, for example a keratin-containing substrate such as hair or skin, a mucous membrane, a mucocutaneous tissue, or a textile substrate, for example a natural or synthetic textile, fabric, fiber, or other surface such as a clothing item or personal accessory. The coloring composition is suitably a hair coloring composition, in particular for coloring a user's head of hair. In other cases, the hair coloring composition can be used to color any hair on a user's body, for example on the user's face (e.g., beard, eyebrows), arms, legs, chest, back, etc.

When the substrate is hair, the target portion of hair may be wet or dry and may applied by a hair professional or at home. In one example, it may be desirable to wash the hair, but not condition it, and then dry the hair before applying the hair coloring composition. In this example, after applying the hair coloring composition, the hair coloring composition is allowed to remain on the hair, optionally covered (e.g., with a shower cap or the like), for at least 10 minutes (e.g., 15, 20, 25, 30, or even up to 60 minutes) before rinsing the hair with water. The hair may then be irradiated with an activating EMR, such as one of the EMR types described herein, followed by irradiation with a second (or more) activating EMR and/or one or more deactivating EMR, such as one of the deactivating EMIR described herein.

In some instances, it may be desirable to activate and/or deactivate the photochromic compound(s) in the coloring composition prior to applying it to hair or another substrate. For example, a coloring composition including two or more photochromic compounds may be placed in a transparent or translucent container and exposed to an activating EMR to change at least one of the compounds to a different color. The activated or partially activated hair dye composition may then be applied to the substrate. Additionally or alternatively, the coloring composition may be exposed to a second activating EMR and/or first deactivating EMR to provide a second color and prior to application to the hair. Further, the activated hair dye composition may be exposed to an additional activating EMR or deactivating EMR after application to hair. This process may be repeated as needed to achieve a desired hair color.

A general method according to the disclosure for reversibly or permanently coloring a substrate (e.g., hair, textile, or otherwise) includes applying a photochromic compound or a coloring composition including the photochromic compound to a target portion of a substrate having a first color, optionally activating the photochromic compound or coloring composition on the target portion before, after, or both before and after application, and optionally (partially) deactivating the photochromic compound or coloring composition on the target portion. Activation can include irradiating the coloring composition with EMR having a wavelength of 250 nm to 395 nm. Activation of the photochromic compound is optional in the sense that the photochromic compound (or coloring composition containing same) as provided to the user could have been previously activated such that it has a color other than that of its base or unactivated state. Even when the composition initially contains some activated form of the photochromic compound, the composition can be further activated before or after application to adjust the color. When the coloring composition does not initially substantially contain an activated or colored form of the photochromic compound (e.g., second or third fluorine-free compound; Formula (I) activated analog or dyotropic rearrangement), the composition is activated to impart a color to the composition and corresponding substrate. In any case, after application of the compound or composition to the substrate, and after any activation that is performed, the target portion of the substrate has a second color (e.g., different from the first or initial color of the substrate). In some cases, the photochromic compound or coloring composition can be at least partially deactivated, for example before and/or after application and/or activation, such that the target portion of the substrate has a third color (e.g., different from the first and/or second colors of the substrate). Deactivation can be performed to adjust or tune the initial color provided after activation, for example by adjusting intensity or selectively targeting a subset of photochromic compounds in the composition. In other cases, deactivating EMR can be applied to fully deactivate the photochromic compound, for example with sufficient time and intensity to rapidly return the first or initial color, such as within about 1, 2, 5, 10, 20, 50, 100, 200, 300 seconds or ranges therebetween. Deactivation can include irradiating the coloring composition with EMR having a wavelength of 400 nm to 1400 nm.

There are three illustrative embodiments of the general coloring method. In a first embodiment, the coloring composition initially contains an activated form of the photochromic compound before application, and the coloring composition need not be further activated before of after application, but such further activation is possible to enhance or adjust the color. For example, coloring composition can be packaged and sold in an opaque container with the photochromic compound in a pre-activated form so that it can be applied by a user without activation. In a second embodiment, the coloring composition initially contains the unactivated form of the photochromic compound before application (e.g., substantially only the unactivated form is present), and the coloring composition is activated before being applied to the target portion of the substrate. For example, the coloring composition can be packaged and sold with the photochromic compound in an unactivated form so that it can be activated and then applied by a user. In a third embodiment, the coloring composition initially contains the unactivated form of the photochromic compound before application (e.g., substantially only the unactivated form is present), and the coloring composition is activated after being applied to the target portion of the substrate. For example, the coloring composition can be packaged and sold with the photochromic compound in an unactivated form so that it can be activated on the substrate after being applied by a user. Regardless of the particular embodiment, the coloring composition as initially provided and prior to application can include the photochromic compound in any relatively proportions for its forms, including its unactivated base form (e.g., first fluorine-free compound or Formula (I) compound), activated analog form (e.g., second fluorine-free compound or activated analog of Formula (I)), and dyotropic rearrangement form (e.g., third fluorine-free compound or dyotropic rearrangement of Formula (I)), as described above.

The first, second, and third colors of the substrate (e.g., first, second, and third hair colors when the substrate is hair) are visibly distinguishable from each other. For example, ΔE between the first color and the second color can be at least 2, a ΔE between the first color and the third color according can be at least 2, and a ΔE between the second color and the third color can be at least 2, where ΔE is determined according to the Color Measurement method. More generally, the ΔE between any two colors can be at least and/or up to 1, 2, 3, 4, 5, 7, 10, 15, 20, 30, 40, or 50 and ranges therebetween. Alternatively or additionally the first, second, and third colors of the substrate can have different or distinct HSL values relative to each other, with the general ranges of H=190° to 360°, S=2.6% to 100%, and L=6.5% to 67% according to the Color Measurement method. Similar ranges and color differences can apply when additional application, activation, and/or deactivation steps are performed to provide fourth, fifth, sixth, etc. colors of the substrate.

The disclosed method can be used to reversibly or permanently color a substrate, depending on the time and intensity of activating EMR as well as the particular photochromic compound being activated. As described above, the activated analog form of the photochromic compound (e.g., fluorine-free second compound) is a reversible state that can return to the unactivated or base form of the photochromic compound (e.g., fluorine-free first compound) with application of visible or infrared light (FIG. 1 left and middle). In contrast, however, the dyotropic rearrangement form of the photochromic compound (e.g., fluorine-free third compound) is an irreversible, stable state that will remain even in presence of deactivating EMR (FIG. 1 middle and right). Accordingly, when a reversible color change is desired, the coloring composition can be activated form a time (and at an intensity/fluence) sufficient to form the fluorine-free second compound, but without substantial formation of the fluorine-free third compound. Suitable activation times for reversible color change can vary depending on the particular activation kinetics of a given compound and/or whether partial or full activation is desired, but it typically can range from 1-600 seconds or 30-300 seconds, for example at least and/or up to 1, 2, 5, 10, 20, 30, 45, 60, 90, 120, 180, 240, 300, 450, or 600 seconds and ranges therebetween. Alternatively, when an irreversible color change is desired, the coloring composition can be activated form a time (and at an intensity/fluence) sufficient to form the fluorine-free third compound. For a given light source wavelength and intensity, a longer period of activation is required to reach the irreversible color state. Using the time required to achieve near color saturation of the photochromic compound in its reversible activated analog form (fluorine-free second compound) as a reference, for example at least and/or up to 75, 85, 95, or 100% of maximum color intensity for the activated analog form, typically a factor of time to reach its irreversible dyotropic rearrangement form is at least and/or up to 1.5, 2, 2.5, 3, 5, 7, 10, and ranges therebetween. For example, if 5-minute activation time with a given light source is sufficient to achieve at least 75% color intensity of the reversible activated analog form, then a 10-minute (or more) activation time with the same light source is typically sufficient to achieve the irreversible dyotropic rearrangement form (e.g., a 2× time factor). In embodiments, suitable activation times for irreversible color change can vary depending on the particular activation kinetics of a given compound, but it typically can range from 20-2400 seconds or 60-1200 seconds, for example at least and/or up to 20, 30, 45, 60, 90, 120, 180, 240, 300, 600, 900, 1200, 1800, or 2400 seconds and ranges therebetween. Regardless of whether the EMR is being applied for reversible or irreversible activation (or even for deactivation), the fluence of the EMR can range from fluence of 1 mJ/cm² to 50 J/cm², for example at least and/or up to 0.001, 0.01, 0.1, 1, 2, 5, 10, 20, 30, 40, or 50 J/cm², and ranges therebetween.

As described above, the photochromic compounds according to the disclosure can be characterized by their relatively long deactivation times (t_{1/2d}) in ambient light settings, making them particularly useful for changing the color of a substrate to be displayed in a natural or artificial light setting during its normal use, for example a hair color design that is intended to be seen in an outdoors/natural light setting or an indoors/artificial light setting. The long-lasting nature of this reversible color change when the photochromic compound is applied to a substrate can be reflected in the length of time that activated color of the substrate remains substantially the same as or close to its activated color. In embodiments, after activation to the second color, the target portion of the substrate can be characterized by a ΔE of not more than 1, 2, 3, 4, or 5 at 2, 4, 6, 8, 10, 12, or 24 hours after activation. For example, after naturally fading in ambient light conditions (e.g., the same ambient light test conditions to determine t_{1/2d} described below), the ΔE between the substrate second color upon initial activation and the substrate faded color after the prescribed time is not more than 1, 2, 3, 4, or 5. Similar characterizations and ranges can apply to the third color when there is also an initial partial deactivation to tune the color, fourth etc. colors when multiple activations and/or deactivations are performed, etc.

### Coloring Products

The coloring compositions (or hair coloring compositions) herein may be placed in a primary and, optionally, a secondary package and displayed in a retail environment for sale. The material(s) used to make the primary and/or secondary packaging is not particularly limited and can include any suitable material known for use in hair dye packaging. In some aspects, it may be desirable to use sustainably sourced and/or recyclable materials. The package may include indicia that communicate to a consumer that the hair coloring product is a tunable hair coloring product. For example, the package may depict hair with its natural color and the hair with two or more other colors provided by the hair coloring composition. In some aspects, the packaging may include indicia that show a spectrum of hair colors provided the hair dye composition as a function of EMR exposure time and/or fluence.

In embodiments, the secondary package can be in the form of a box. The secondary package is configured to house a primary package containing a coloring composition. The primary package may be in the form of a bottle, jar, vial or other package suitable for containing the coloring composition. In some aspects, a primary package and/or coloring composition may be visible through a window in the secondary package. In some aspects, it may be desirable to inhibit or prevent EMR from reaching the hair dye composition, for example, by configuring the primary and/or secondary packages to block EMR that can activate and/or deactivate a hair dye composition contained in the primary packaging (e.g., EMR having a wavelength of 200 to 1400 nm, 200 to 700 nm, 250 to 700 nm, 400 to 1400 nm, 400 to 700 nm, or 280 to 400 nm). In embodiments, the secondary package may include indicia that communicate the tunable aspect of the coloring composition, for example indicia including series of colored shapes or labels with colors corresponding to the color of the composition after a duration of EMR activating or deactivating exposure corresponding to the label.

In some aspects, the coloring composition may be provided as a kit that includes two or more photochromic compounds and/or photochromic coloring compositions that can be combined by a user to form a coloring composition mixture that provides a desired color for hair or other substrate to which the composition is applied. In some aspects, the kit includes two or more coloring compositions, each including a photochromic compound, a suitable solvent, and, optionally, other ingredients commonly included in a coloring composition. In some aspects, the kit includes two or more photochromic compound powders that can be combined before or after dissolving the powder in a suitable solvent or carrier. The photochromic compound powders and/or hair coloring compositions in the kit can be combined at different ratios to provide a variety of different hair colors. The kit may also include a mixing container for combining the photochromic compound powders and/or coloring compositions. The photochromic compounds may be activated and/or deactivated before and/or after forming a coloring composition mixture. The kit may also include a light source to provide activating or deactivating EMR. In embodiments, the light source and the container containing the coloring composition can be sold separately, for example being spatially separated from each other in a retail environment while still displaying a common brand.

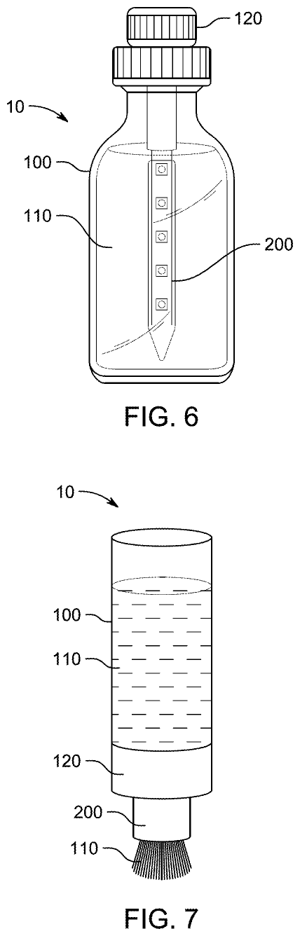

FIGS. 6-10 illustrate several embodiments of products (or systems) 10 for reversibly or irreversibly coloring a substrate (e.g., hair, textile, or otherwise). The product 10 includes a container (or primary packaging) 100, which hold or contains the coloring composition 110 therein. As described above, the composition 110 includes one or more photochromic compounds in any distribution of forms (e.g., unactivated, activated analog, dyotropic rearrangement), optionally mixed or blended with one or more solvents, other carrier components, and/or other optional ingredients. The container 100 is not particularly limited, and it can include any suitable vessel such as a jar, tube, cannister, etc. formed from glass, plastic, metal, etc. to hold the composition 110 in any suitable form such as liquids/solutions, suspensions, shampoos, conditioners, lotions, creams, gels, sticks, sprays, aerosols, ointments, solid bars, pastes, foams, mousses, hydrogels, and the like. In embodiments, the container 100 can be or include a refillable and/or replaceable cartridge containing the composition 110 therein. The product 10 also includes a light source 200 adapted to provide activating EMR (e.g., only activating EMR), deactivating EMR (e.g., only deactivating EMR), or both activating and deactivating EMR (e.g., at alternating points in time). In illustrative embodiments, the light source 200 can be incorporated into the product container 100 to activate/deactivate the photochromic compound in the container 100 or as it is being applied from the container 100, or the light source 200 can be separate from the product container 100 (e.g., a brush with integrated light source) and can be used to activate/deactivate the photochromic compound after application to a substrate such as a user's hair or otherwise. The light source 200 is not particularly limited, for example including a lamp, LED, laser, etc. The light source 200 can have a fixed or adjustable wavelength (or range of wavelengths) of emitted light, a fixed or adjustable power (or range of powers) of emitted light, and/or a fixed or adjustable intensity (or range of intensities) of emitted light. In embodiments, the light source 200 can be adapted to provide one or both of activating EMR having a wavelength of 250 nm to 395 nm and deactivating EMR having a wavelength of 400 nm to 1400 nm. In embodiments, the product 10 can further include a secondary package (not shown) to house and/or display the container 100, the composition 110 therein, and/or the light source 200.

The coloring composition 110 can include the photochromic compound in any of its forms, including its unactivated base form (e.g., first fluorine-free compound or Formula (I) compound), activated analog form (e.g., second fluorine-free compound or activated analog of Formula (I)), and dyotropic rearrangement form (e.g., third fluorine-free compound or dyotropic rearrangement of Formula (I)). Each form can independently be absent, substantially absent, or present in any amount relative to all forms combined, for example with each form independently being present in an amount of at least and/or up to 0, 1, 2, 5, 10, 20, 25, 30, 40, 50, 60, 70, 75, 80, 90, 95, 98, 99, or 100 mol. % and ranges therebetween relative to all three forms. In embodiments, the coloring composition 110 includes the activated analog form and/or the dyotropic rearrangement form of the photochromic compound, for example with a minor amount or substantially no unactivated form. In embodiments, the coloring composition 110 includes the unactivated form of the photochromic compound, for example with a minor amount or substantially no activated analog form and/or dyotropic rearrangement form.

In embodiments, the container 100 is opaque. For example, the container 100 can be completely opaque, having opaque walls, cap, applicator, etc. to prevent exposure to deactivating ambient light and maintain the compound in activated form during the product 10 lifetime of packaging, storage, shipping, display, etc.

In embodiments, the container 100 is at least partially transparent. For example, the container 100 can include a bottle wall or other structure with a window or transparent section to permit external viewing of the composition 110 color, for example when the light source 200 is used to activate the compound inside the container 100.

FIG. 6 illustrates an embodiment of the product 10 in which the light source 200 is associated with the container 100 interior and is adapted to irradiate the coloring composition 110 while it is in the container 100 (e.g., prior to the composition 110 being dispensed or applied). The light source 200 can be permanently or removably attached to the container 100, such as an exterior wall-mounted light source, an applicator cap/brush-mounted light source, etc. As particularly illustrated, the light source 200 can be attached to or integrated with a cap 120 that can include a switch to activate the light source 200 and that can be removed to dispense or apply the composition 110 onto a substrate (not shown).

FIG. 7 illustrates an embodiment of the product 10 in which the light source 200 is associated with the container 100 exterior and is adapted to irradiate the coloring composition 110 external the container 100 (e.g., after or while the composition 110 is being dispensed or applied). The light source 200 can be permanently or removably attached to the container, such as an exterior wall-mounted light source, an applicator cap/brush-mounted light source, etc. As particularly illustrated in FIG. 7, the container 100 can be a clear/transparent material that includes a cap 120 with an applicator and integrated light source 200 so that the composition 110 can be activated by the light source 200 as it is being dispensed from the container 100

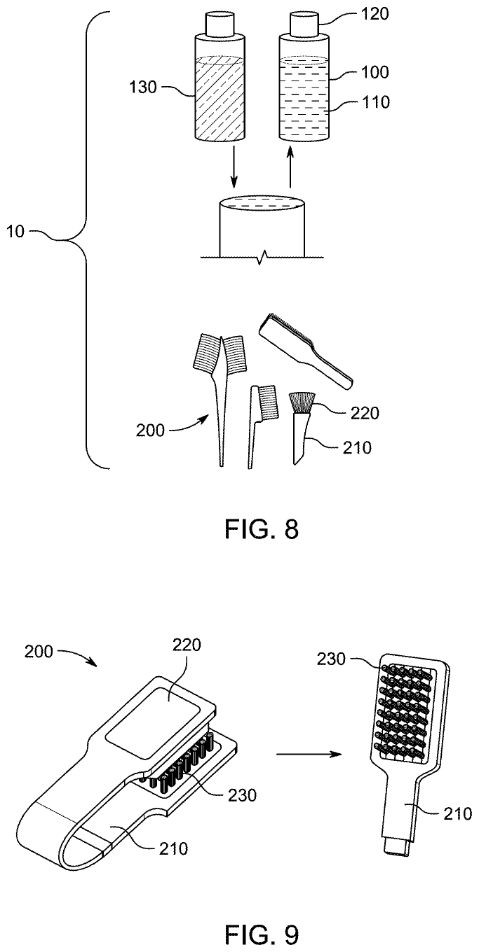

FIGS. 8-9 illustrate embodiments of the product 10 in which the light source 200 is separate from the container (e.g., not physically attached and/or attachable to the container) and is adapted to irradiate the coloring composition 110 external to the container 100 (e.g., after or while the composition 110 is being dispensed or applied). In embodiments, the light source can be in the form of a brush or comb 210, such as a hair brush or comb, a makeup brush or comb, etc. A lamp 220 of the light source 200 can be integrated into a base or handle from which the bristles or teeth 230 extend, or the lamp 220 of the light source 200 can be integrated into a bristle-opposing surface, such as in a clamp or clamshell configuration. As particularly illustrated in FIG. 8, the container 100 can be a clear/transparent material that includes a cap 120 with an applicator, and the container 100/composition 110 can be paired with an additional hair care composition 130 (e.g., shampoo, conditioner, etc.) as part of the product 10 for use in combination with the composition 110. As further illustrated, the light source 200 can include one or more brushes or combs 210 adapted to emit the EMR from the lamp 220 of the light source 200 at or near bristles or teeth 230 so that color tuning can be performed while also brushing or combing hair to which the composition 110 is applied. FIG. 9 illustrates an alternative embodiment in which the light source 200 has a clamp or clamshell configuration with a lamp 220 portion opposing a brush 210 portion with bristles or teeth 230. The lamp 220 and brush 210 portions of the light source 200 as removably joined such that color tuning can be performed while also brushing or combing hair to which the composition 110 is applied (i.e., when the two components 210, 220 are joined; FIG. 9 left), and the brush 210 can be subsequently disconnected from the lamp 220 (FIG. 9 right) for further brushing after activation or deactivation is complete.

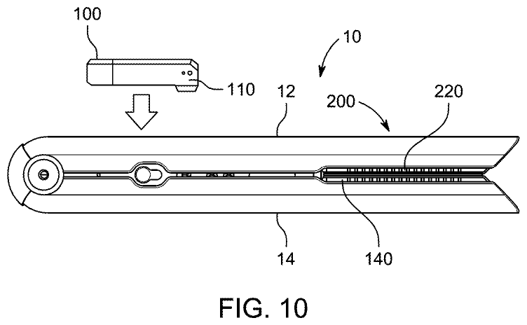

FIG. 10 illustrates an embodiment of the product 10 in which the light source 200 is associated with the container 110 or product 10 exterior and is adapted to irradiate the coloring composition 110 external to the container 100 (e.g., after or while the composition 110 is being dispensed or applied). The product 10 has a clamp or clamshell structure with a first surface 12 incorporating a lamp 220 of the light source 200, and an opposing second surface 14 incorporating a color deposition plate 140. The container 110 is in the form of a refillable and/or replaceable cartridge that can be inserted into the product 10 body (e.g., in the first or second surface 12, 14 thereof). The container 110 is in fluid communication with the color deposition plate 140 such that the composition 110 can be dispensed (e.g., via applied pressure) through the plate 140 to hair (not shown) between the plate 140 and lamp 220, at which time the hair can be activated or deactivated via the lamp 220.

### Color Measurement Method

This method enables the measurement of both the initial color and color change on hair, textile, or other substrate treated with the coloring compositions herein. The method uses a suitable spectrophotometer or colorimeter such as an X-Rite Ci7800 sphere benchtop brand spectrophotometer, Konica Minolta CM-3600A brand spectrometer or equivalent according to the manufacturer's instructions. The value used to express the degree of color change on any particular substrate is Delta E (ΔE), which is calculated using L*, a* and b* values (“Lab values”) according to Equation 1 below. In Equation 1, L₁ is the first L* value and L₂ is the second L* value; a₁ is the first a* value and a₂ is the second a* value; and b₁ is the first b* value and b₂ is the second b* value. The first and second values can represent the values before and after a color change, respectively, for example including an initial uncolored (or natural) state and a subsequent colored (or activated) state, or an initial colored (or activated) state and a subsequent color-adjusted (or further activated/deactivated) state. L* is a measure of lightness and darkness (color intensity), wherein L=100 is white, and L=0 is black. The a* value is a measure of the red and green quotients (color hues) in which positive values equate to red and negative values equate to green. The b* value is a measure of the yellow and blue quotients (color hues) in which positive values equate to yellow and negative values equate to blue.

$$Δ E = {[ {( {L}_{1} - {L}_{2} )}^{2} + {( {a}_{1} - {a}_{2} )}^{2} + {( {b}_{1} - {b}_{2} )}^{2} ]}^{0.5} \qquad ( 1 )$$

The Lab value measurements are carried out on a full scanning spectrophotometer with a wavelength of from 400-700 nanometers. The spectrophotometer is set to: mode=SCI/SCE; spot size=10 mm; and light=D65. The spectrophotometer records the color in terms of L* a* and b* values, which can be converted into other color systems (such as the HSL color system) using known conversion techniques.

When the sample is a hair tress (e.g., standard hair switch), the sample is placed in a holder designed to hold the hair sample in a uniform orientation during measurement and ensure it does not move during measurement. For example, the hair switch may be place on a hard, flat surface (e.g., board) and secured at the top and bottom to prevent movement. In some instances, it may be desirable to comb the hair sample prior to securing it so it lies flat on the surface. The hair should be spread to cover the 10 mm port of the spectrophotometer during color measurement. Three measurements are run per treatment.

Color and color change can also be measured using absorbance and changes in absorbance. Absorbance can be measured using any suitable spectrophotometer. Compounds of the disclosure and their activated analogs have characteristic absorbance spectra. Absorbance of a compound in the unactivated state is, in general, different from absorbance of activated analogs of the compound.

Compounds of the disclosure can be characterized by peaks in their respective absorbance spectra (generally, λₘₐₓ). For instance, compounds of the disclosure can be characterized by a characteristic activation wavelength (λₐ) and a characteristic deactivation wavelength (λ_{d}), corresponding to peaks in the absorbance spectra of the unactivated and activated states, respectively.

### Activation and Deactivation Times

A 50% activation time for a compound of the disclosure was determined by continuously irradiating a solution of the compound with an activating EMR, periodically measuring absorbance of the sample at λ_{d} (i.e., the peak wavelength in the absorbance spectrum of the corresponding activated analog of the compound) to obtain a plot of absorbance vs. time, determining the maximum absorbance of the sample (i.e., upon complete conversion to the activated analog), and determining the time at which the sample exhibited half of the maximum absorbance.

Similarly, a 50% deactivation time for a compound of the disclosure was determined by continuously irradiating a solution of the activated analog of the compound with a deactivating EMR, periodically measuring absorbance of the sample at l_{d} of the activated analog to obtain a plot of absorbance vs. time, and determining the time at which the sample exhibited half of its initial absorbance.

In the examples below, solutions of the synthesized molecules in isopropyl myristate (IPM) at a concentration of 0.1 mM were prepared. For color activation (open-to-closed ring isomer as the reversible activated analog), 3.0 mL of the solution was transferred into a quartz cuvette (Hellma Analytics High Precision Cell, 10 mm light path) and exposed to ultraviolet (UV) radiation (Spectroline, ENF-240C/FE; UVA lamp with 1×365 nm 4-Watt Tube and 1×254 nm 4-Watt Tube) at a distance of 2.54 cm. UV-Vis measurements were taken at 0, 0.5, 1.0, 3.0, and 5.0 minutes after UV exposure. For color deactivation (closed-to-open ring isomer), 3.0 mL of the solution was transferred into a quartz cuvette and exposed to UV radiation for 10.0 minutes under the same conditions. The colored solutions were then illuminated with white light (Koncept LED desk lamp, 5000 K) positioned 50.8 cm away for 0, 0.5, 4, and 8 hours. The white light intensity for the ambient light decay test is 5 W/m² (tested using Daystar DS-05 solar meter, Raydec, Inc.) UV-Vis measurements were recorded at these time points. For irreversible color activation (dyotropic rearrangement form), the molecules prepared in isopropyl myristate (IPM) at a concentration of 0.1 mM are exposed to UV radiation (Spectroline, ENF-240C/FE; UVA lamp with 1×365 nm 4-Watt Tube and 1×254 nm 4-Watt Tube) for at least 10.0 minutes or twice the duration to reach full reversible activation, whichever is longer. For both activation and deactivation, additional and/or alternate time points for measurement can be selected appropriate for a given compound (e.g., for photochromic compounds with relatively longer or shorter activation or deactivation times, etc.). The absorption spectra of the synthesized molecules in IPM were analyzed using a UV-Vis spectrophotometer (UV-2700, Shimadzu, Japan). Measurements were performed with a 3.0 mL sample in a quartz cuvette, scanning from 190 nm to 800 nm with a 1.0 nm step size and a 2.0 nm slit width.

Data fitting: The intensity-activation (or deactivation) time plot was generated by harvesting the maximum absorption intensity points from the absorption spectra collected at different time intervals during activation (or deactivation). The activation (or deactivation) plot was fitted using a suitable growth (or decay) function. A few growth models used for the fitting are Sigmoid/logistic (for fast activation) and Michalis-Menten saturation models, while exponential decay function was use for the decay plot. The fitting was reiterated by optimizing the fitting parameters and changing the fitting models to achieve high R-squared value (as close as possible to 1). In general, any suitable curve-fitting function can be used which has an R-squared value of at least 0.98, 0.99, or 0.995.

### Phototoxicity and Photoreactivity

Compounds according to the disclosure were evaluated for phototoxicity according to OECD 432 (in vitro 3T3 NRU phototoxicity test), which assesses phototoxicity potential of a substance by comparing cytotoxicity of the substance in the presence and absence of radiation.

Compounds according to the disclosure were evaluated for phototoxicity according to OECD 495 (reactive oxygen species assay for photoreactivity), which measures generation of reactive oxygen species under simulated sunlight conditions.

Both methods are known to the skilled person and are electronically available from the OECD (Organisation for Economic Co-operation and Development). Both OECD 432 and 495 recommend the use of analytical grade dimethyl sulfoxide (DMSO) to solubilize the test compounds. In some cases, due to the limited solubility of some of the photochromic compounds in DMSO, an alternative organic solvent (e.g., acetone) can be used for the OECD 495 assay. For example, the photochromic compound of Example 3 below was dissolved in acetone to prepare a test solution with a final concentration of 200 μM. Similarly, the photochromic compound of Example 4 below was dissolved in acetone to prepare a test solution with a final concentration of 20 μM. For the photochromic compound of Example 5 below, either DMSO or acetone could be used to prepare the test solution. For the OECD 432 assay, however, because the concentration range is lower (5-100 ug/mL, for example test concentrations at one or more of 5, 10, 20, 25, 50, 75, or 100 ug/mL), DMSO can be used for all the test compounds.

### EXAMPLES

The following examples further describe and demonstrate embodiments within the scope of the present invention. They are given for the purpose of illustration and are not to be construed as limitations of the present invention.

### Examples 1-9—Photochromic Compounds

Structures of Examples 1-9, photochromic compounds according to the disclosure, are shown in Table 2. Syntheses of example compounds and related intermediates are described below. Briefly, intermediates DAE-1 and then DAE-2 are synthesized, and then each of Examples 1-5 are synthesized by functionalizing DAE-2 with the indicated substituted aromatic group. Scheme 1 below illustrates the synthetic pathway for the examples, including the DAE-1 intermediate (top right), the DAE-2 intermediate (bottom right), and the photochromic compound product (bottom left).

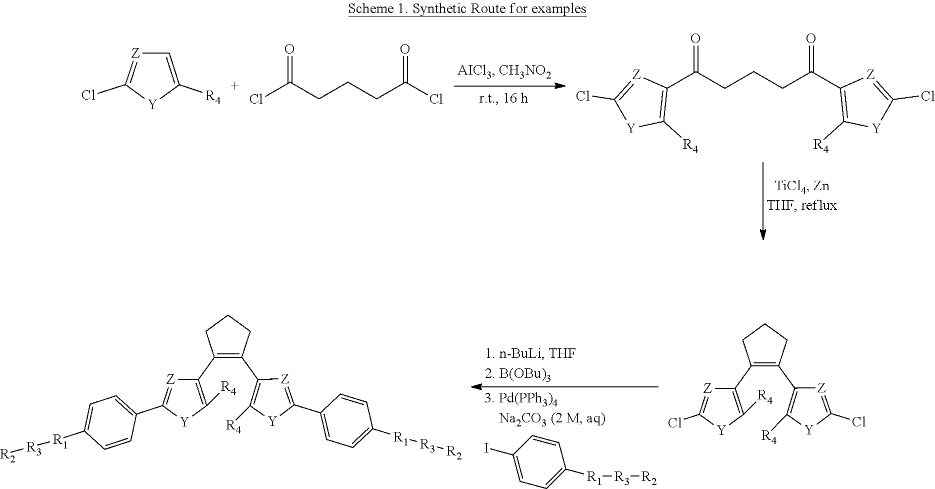

TABLE 2
Photochromic Compounds

| Example | 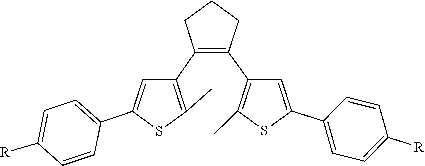 |
|---|---|
| 1 | R = C(═O)O(CH₂CH₂O)₃CH₃ |
| 2 | R = O(CH₂CH₂O)₃CH₃ |
| 3 | R = OC₁₄H₂₉ |
| 4 | R = C(═O)OC₁₄H₂₉ |
| 5 | R = OC(═O)C₁₁H₂₃ |
| 6 | R = OCH₃, plus an additional OCH₃ group |
| | on each aromatic ring adjacent to R |
| 7 | R = C(═O)OC₂H₅ |
| 8 | R = C(═O)OH |
| 9 | R = OH, plus an additional OH group on |
| | each aromatic ring adjacent to R |

Synthesis of 1,5-bis(5-chloro-2-methylthiophen-3-yl)pentane-1,5-dione (DAE-1). Under fast stirring of an ice-cooled solution of dry AlCl₃ powder (15.6 g, 117.00 mmol) in nitromethane (150 ml), 2-chloro-5-methylthiophene (1) (15.60 g, 117.64 mmol, 12.9 mL) and glutaryl chloride (2) (9 g, 53.25 mmol, 6.8 mL) were successively dropwise added. After addition, the reaction mixture was stirred for 16 h at room temperature, and the color turned to dark red. Then a mixture of concentrated HCl (100 mL) and ice (150 g) was carefully added to the reaction mixture, and the resulting aqueous layer was extracted with dichloromethane. The combined organic phases were successively washed with saturated aqueous NaHCO₃ solution, water, and saturated aqueous NaCl solution, then dried over MgSO₄ and filtered. The solvent was evaporated in vacuo to yield DAE-1 as a brown tar (18.6 g, 96%).

¹H NMR (400 MHz, CDCl₃): δ=7.18 (s, 2H), 2.86 (t, J=6.9 Hz, 4H), 2.66 (s, 6H), 2.06 (p, J=7.0 Hz, 2H).

Synthesis of 1,2-bis(5-chloro-2-methylthiophen-3-yl)cyclopentene (DAE-2). TiCl₄ (111 mL, 111 mmol, 1 M solution in CH₂Cl₂) was added cautiously to ice-cooled dry THF (160 mL) under N₂ atmosphere. To the mixture was added Zn powder (8.69 g, 132.9 mmol) and the resulting mixture was stirred for 2 h at 65° C. Afterwards, the reaction was cooled to 0° C. with an ice bath, and DAE-1 (16.0 g, 44.3 mmol) was added. The resulting mixture was heated at 65° C. for 4 h. Several drops of saturated aqueous K₂CO₃ solution were added to the mixture, followed by filtration over Celite and washing with ethyl acetate, and the filtrate was dried over anhydrous MgSO₄. The crude product was purified by column chromatography (silica, hexanes) to afford DAE-2 as a light yellow solid (9.3 g, 64%).

¹H NMR (400 MHz, CDCl₃): δ=6.58 (s, 2H), 2.72 (t, J=7.5 Hz, 4H), 2.02 (quin, J=7.5 Hz, 2H), 1.89 (s, 6H).

Detailed Synthesis of Example 3. As illustrated in FIG. 2 and described in more detail below, intermediates (5) and then (7) were synthesized, and then DAE-2 was reacted with intermediate (7) to form Example 3.

Synthesis of tetradecyl p-toluenesulfonate (5). 1-Tetradecanol (3) (20 g, 93.29 mmol) was dissolved in pyridine (40 mL). p-Toluenesulfonyl chloride (4) (21.34 g, 111.94 mmol) in 40 mL pyridine was added dropwise and the mixture was stirred at room temperature for 4 h. The reaction mixture was then extracted with CH₂Cl₂ and wished with 2 M HCl aqueous solution. The organic layer was dried over anhydrous MgSO₄, filtered and concentrated. The obtained oil was subjected to column chromatography (ethyl acetate:hexames=1:10) to give tetradecyl p-toluenesulfonate (5).

¹H NMR (400 MHz, CDCl₃): δ=7.79 (d, J=8.3 Hz, 2H), 7.34 (d, J=8.0 Hz, 2H), 4.01 (t, J=6.5 Hz, 2H), 2.44 (s, 3H), 1.62 (p, J=6.6 Hz, 2H), 1.41 (p, J=6.9 Hz, 2H), 1.25 (s, 18H), 0.88 (t, J=6.7 Hz, 3H).

Synthesis of 1-iodo-4-(tetradecyloxy)benzene (7). Tetradecyl p-toluenesulfonate (5) (8.57 g, 23.25 mmol) and 4-iodophenol (6) (5.63 g, 25.58 mmol) were added to a solution of K₂CO₃ (19.28 g, 139.51 mmol) in DMF (50 mL). The solution was stirred for 16 h at 70° C. After cooling, the mixture was poured into aqueous HCl (pH=2) and extracted with CH₂Cl₂ (100 mL×2). The organic layer was washed with water (100 mL×3) and brine (100 mL×1) and dried over MgSO₄, and the solvent was evaporated in vacuo. The crude product was purified by silica gel column chromatography (hexane) to yield 1-iodo-4-(tetradecyloxy)benzene (7) (6.8 g, 70%) as a white solid.

¹H NMR (400 MHz, CDCl₃): δ=7.53 (d, J=8.8 Hz, 2H), 6.67 (d, J=8.8 Hz, 2H), 3.91 (t, J=6.6 Hz, 2H), 1.80-1.72 (m, 2H), 1.43 (p, J=6.9, 6.5 Hz, 2H), 1.26 (s, 20H), 0.88 (t, J=6.7 Hz, 3H).

Synthesis of 1,2-bis(2-methyl-5-(4-(tetradecyloxy)phenyl)thiophen-3-yl)cyclopent-1-ene (Example 3). DAE-2 (1 g, 3.04 mmol) was dissolved in anhydrous THF (10 mL), and n-BuLi (3.8 mL of 2.0 M solution in cyclohexane, 7.6 mmol) was injected slowly under nitrogen atmosphere at 0° C. The reaction mixture was brought to room temperature and stirred for a further 30 min. Then tri-n-butylborate (2.05 mL, 1.75 g, 7.59 mmol) was added in one portion to the solution. The resulting reddish solution was stirred at room temperature for 1 h. The resulting bis(boronic acid) mixture was used in the following Suzuki cross coupling reaction without further purification.

Pd(PPh₃)₄ (175 mg, 0.15 mmol) was added to a solution of 1-iodo-4-(tetradecyloxy)benzene (7) (3.16 g, 7.59 mmol) in THF (20 mL). The mixture was stirred for 15 min at room temperature. under nitrogen atmosphere. Then aqueous Na₂CO₃ (15 mL, 2 M) were added to the resulting solution. The mixture was heated under reflux at 70° C., and the bis(boronic ester) mixture obtained above was added dropwise by a syringe. Then the solution was refluxed overnight and cooled to the room temperature. After that, H₂O was added and extracted with diethyl ether. The collected organic layers were dried over Na₂SO₄ and, after evaporation of the solvent, the compound was purified by column chromatography on silica (ethyl acetate:hexanes=1:20) and the collected product was evaporated and redispersed in hexane to give Example 3 (2.3 g, 91%).

¹H NMR (400 MHz, CDCl₃): δ=7.42 (d, J=8.8 Hz, 4H), 6.93 (s, 2H), 6.87 (d, J=8.8 Hz, 4H), 3.96 (t, J=6.6 Hz, 4H), 2.84 (t, J=7.4 Hz, 4H), 2.08 (quin, J=7.5 Hz, 2H), 1.99 (s, 6H), 1.83-1.75 (m, 4H), 1.46 (td, J=9.8, 8.7, 4.7 Hz, 4H), 1.28 (s, 40H), 0.92-0.88 (m, 6H).

¹³C NMR (100 MHz, CDCl₃): δ=158.55, 139.73, 136.69, 134.70, 133.47, 127.41, 126.67, 123.01, 114.92, 100.04, 68.25, 38.62, 32.08, 29.84, 29.81, 29.75, 29.73, 29.55, 29.52, 29.41, 26.18, 23.16, 22.84, 14.49, 14.27.

Detailed Synthesis of Example 4. As illustrated in FIG. 3 and described in more detail below, intermediate (9) was synthesized, and then DAE-2 was reacted with intermediate (9) to form Example 4.

Synthesis of tetradecyl 4-iodobenzoate (9). Into a 250 mL round-bottom flask, 4-iodobenzoic acid (8) (14 g, 56.45 mmol), 1-tetradecanol (3) (14.52 g, 67.74 mmol), 1-ethyl-3-(3-dimethylaminopropyl)carbodiimide (EDCI; 13 g, 67.74 mmol), (4-dimethylamino)pyridine (DMAP; 3.45 g, 28.22 mmol), and anhydrous CH₂Cl₂ (200 mL) were charged. The mixture was stirred at room temperature for 12 hours. The mixture was purified by silica gel column chromatography (ethyl acetate:hexanes=1:20). After removing the solvents, the final product 9 was obtained as a white solid in quantitative yield (25 g, 100%).

¹H NMR (400 MHz, CDCl₃): δ=7.80 (d, J=8.5 Hz, 2H), 7.74 (d, J=8.5 Hz, 2H), 4.30 (t, J=6.7 Hz, 2H), 1.79-1.71 (m, 2H), 1.41 (dd, J=15.6, 7.3 Hz, 2H), 1.26 (s, 21H), 0.88 (t, J=6.8 Hz, 3H).

Synthesis of 1,2-bis(2-methyl-5-(4-(tetradecylcarboxy)phenyl)thiophen-3-yl)cyclopent-1-ene (Example 4). DAE-2 (1.0 g, 3.04 mmol) was dissolved in 10 mL of anhydrous THF, and n-BuLi (2.0 M in cyclohexane, 3.8 mL, 7.59 mmol) was added dropwise at 0° C. The solution was then stirred and warmed to room temperature for 30 min. Then, tri(n-butyl)borate (2.05 mL, 1.75 g, 7.59 mmol) was added in one portion and the resulting mixture was stirred at room temperature for 1 h. The reddish solution was stirred at room temperature for 1 h. The resulting bis(boronic acid) mixture was then used in the following Suzuki cross coupling reaction without further purification.

Pd(PPh₃)₄ (175 mg, 0.15 mmol) was added to a solution of tetradecyl-4-iodobenzoate (9) (3.37 g, 7.59 mmol) in THF (20 ml). The mixture was stirred for 15 min at room temperature under nitrogen atmosphere. Aqueous Na₂CO₃ (15 mL, 2 M) was added to the resulting solution. The mixture was heated under reflux at 70° C. and the bis(boronic ester) mixture obtained above was added dropwise via syringe. The resulting solution was refluxed overnight and cooled to room temperature. After that, water was added, followed by extraction with diethyl ether. The collected organic layers were dried over MgSO₄, and after evaporation of the solvent, the compound was purified by column chromatography on silica (ethyl acetate:hexanes=1:20). The collected product was evaporated and redispersed in hexane to provide Example 4 (2.1 g, 77%).

¹H NMR (400 MHz, CDCl₃): δ=7.99 (d, J=8.5 Hz, 4H), 7.54 (d, J=8.5 Hz, 4H), 7.14 (s, 2H), 4.31 (t, J=6.7 Hz, 4H), 2.86 (t, J=7.4 Hz, 4H), 2.10 (quin, J=7.6 Hz, 2H), 2.02 (s, 6H), 1.76 (p, J=6.8 Hz, 4H), 1.47-1.41 (m, 4H), 1.26 (s, 40H), 0.88 (t, J=6.8 Hz, 6H).

¹³C NMR (100 MHz, CDCl₃): δ=166.50, 138.69, 137.14, 136.35, 134.88, 130.31, 128.80, 125.52, 124.94, 65.25, 38.62, 32.07, 29.83, 29.79, 29.72, 29.67, 29.50, 29.42, 28.87, 26.18, 23.14, 22.83, 14.69, 14.26.

Synthesis of Examples 1, 2, and 5-7. Examples 1, 2, and 5-7 were synthesized according to procedures similar to those used to prepare Examples 3 and 4, using aryl iodide compounds analogous to (7) and (9) to provide the corresponding phenyl substituents (i.e., R groups in Table 2).

Activation and Deactivation. The absorption spectra of the synthesized molecules in isopropyl myristate (IPM_ were analyzed using a UV-Vis spectrophotometer (UV-2700, Shimadzu, Japan). Measurements were performed with a 3.0 mL sample in a quartz cuvette, scanning from 190 nm to 800 nm with a 1.0 nm step size and a 2.0 nm slit width. Peak absorbance wavelengths of solutions of the open ring (lₐ) and closed ring (l_{d}) forms of Examples 1-6 are shown in Table 3.

TABLE 3
Absorbance Wavelengths

| Example | lₐ | l_{d} |
|---|---|---|
| 1 | 294 nm | 567 nm |
| 2 | 285 nm | 527 nm |
| 3 | 306 nm | 528 nm |
| 4 | 296 nm | 563 nm |
| 5 | 279 nm | 531 nm |
| 6 | 305 nm | 530 nm |

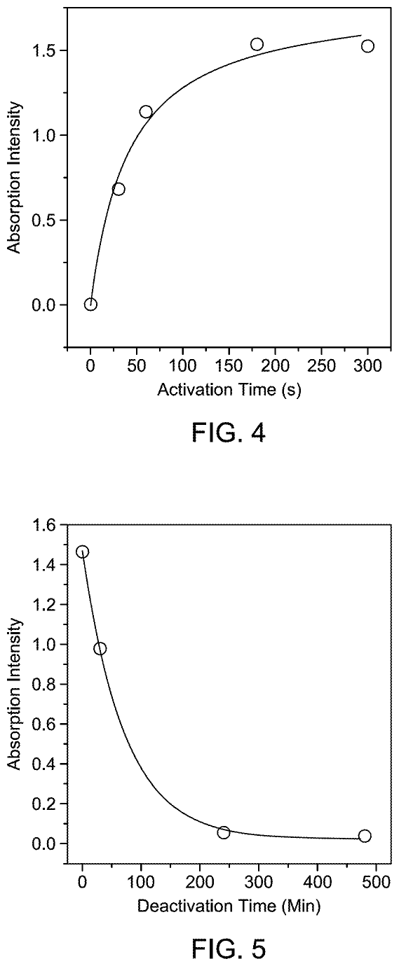

50% activation times and 50% deactivation times for several example compounds were measured according to methods described herein. 0.1 mM solutions of Examples 1-5 were continuously exposed to an activating EMR (365 nm UVA), and absorbance of each solution at l_{d} of the corresponding activated analog as listed in Table 3 was measured periodically during activation. The resulting absorbance vs. time data were used to determine 50% activation times. The solutions were then exposed to a deactivating EMR (white light), and absorbance of each solution at l_{d} of the corresponding activated analog as listed in Table 3 was measured periodically during deactivation. The resulting absorbance vs. time data were used to determine 50% deactivation times. FIGS. 4 and 5 show absorbance vs. time data during activation and deactivation, respectively, of Example 3. Analogous experiments were performed for comparative photochromic compounds DAE-0001 (cyan; 3,3′-(perfluorocyclopent-1-ene-1,2-diyl)bis(2-methyl-5-phenylthiophene)), DAE-0012 (magenta; 4,4′-(perfluorocyclopent-1-ene-1,2-diyl)bis(5-methyl-2-phenylthiazole)). 50% activation and deactivation times are shown in Table 4.

TABLE 4
Activation and Deactivation Times

| | | |
|---|---|---|
| Example | t_{1/2, a} | t_{1/2, d} |

| | | | | |
|---|---|---|---|---|
| 1 | 6.5 | sec | 144.3 | min |
| 3 | 29.8 | sec | 50.2 | min |
| 4 | 12.4 | sec | 148.8 | min |
| 5 | 179.2 | sec | | |
| DAE-0001 | 53.7 | sec | 26.7 | min |
| DAE-0012 | 69.4 | sec | 12.8 | min |

Phototoxicity. Examples 3, 4, and 5 were evaluated for potential phototoxicity according to OECD 432, as described above. No phototoxicity was observed for sample concentrations up to 100 μg/mL (i.e., 100 ppm).

Photoreactivity/Irritation. Examples 3, 4, and 5 were evaluated for potential formation of reactive oxygen species (ROS) according to OECD 495, as described above. The example compounds were found to be non-photoreactive under the test conditions and are accordingly considered to pose low to no risk of photoirritation, photoallergy, or photogenotoxicity.

Solubility. Examples 3, 4, and 5 were evaluated for their solubility (w/w) in isopropyl myristate (IPM) at different temperatures, and the results are shown in Table 5.

TABLE 5
Solubility

| Example | T = 50° C. | T = 40° C. | T = 26-28° C. |
|---|---|---|---|
| 3 | 20% | 20% | 7% |
| 4 | 20% | 20% | 10% |
| 5 | 20% | 10% | <4% |

### Example 10—Sample Formulations

Table 6 shows coloring composition formulas that may be suitable for use as hair coloring compositions incorporating the disclosed photochromic compounds.

TABLE 6
Coloring Compositions

| | Product | |
|---|---|---|
| Composition | Form | Ingredient list |
| 1 | Oil- | Isododecane, dimethicone, dimethiconol, diethyl sebacate, isodecyl |
| | based | pivalate, tris(ethylhexanoate) glyceryl, coconut oil, one or more |
| | | photochromic compounds, fragrance, phenoxyethanol, tocopherol, |
| | | jojoba seed oil |
| 2 | Water | TWEEN80, coconut oil, water, one or more photochromic compounds, |
| | based | fragrance |
| 3 | Mousse | Cyclohexasiloxane, dimethicone, dimethicone/vinyl dimethicone |
| | | crosspolymer, silica, acrylates copolymer, disodium stearoyl |
| | | glutamate, aluminum hydroxide, coconut oil, one or more |
| | | photochromic compounds, fragrance |
| 4 | Solid | Talc, dimethicone, HDI/trimethylol hexyllactone crosspolymer, silica, |
| | powder | dimethicone/vinyl dimethicone crosspolymer, mica, titanium dioxide |
| | | (CI 77891), one or more photochromic compounds, |
| | | triethoxycaprylylsilane, ethylhexylglycerin, glyceryl caprylate, |
| | | dimethiconol, iron oxides (CI 77492), aluminum hydroxide, alcohol, |
| | | butylene glycol, iron oxides (CI 77491), methicone, tocopherol |
| 5 | Soft gel | Water, iota carrageenan, TWEEN80, coconut oil, one or more |
| | | photochromic compounds |
| 6 | Serum | Glycerin, water, dimethicone, butylene glycol, myristyl alcohol, |
| | | propylene glycol, cyclopentasiloxane, isopropyl myristate, cetyl |
| | | ethylhexanoate, hydroxypropyl starch phosphate, perfume, |
| | | steartrimonium chloride, guar hydroxypropyltrimonium chloride, |
| | | sodium benzoate, cetrimonium chloride, laureth-23, laureth-3, |
| | | polyquaternium-7, betaine, milk protein extract, panthenol, hydrolyzed |
| | | collagen, Spirulina platensis extract, Corchorus olitorius leaf extract, |
| | | disodium EDTA, tocopheryl acetate, C12-15 pareth-3, cocamidopropyl |
| | | betaine, hydrolyzed keratin, hydrolyzed collagen PG-propyl |
| | | methylsilanediol, hydrolyzed wheat protein, hydrolyzed corn protein, |
| | | hydrolyzed soy protein, 1,2-hexanediol, glycine, glutamic acid, lysine, |
| | | leucine, methionine, valine, serine, cysteine, aspartic acid, alanine, |
| | | arginine, isoleucine, tyrosine, threonine, phenylalanine, proline, |
| | | histidine, hydrolyzed silk, propanediol, Ceratonia siliqua (carob) fruit |
| | | extract, Diospyros kaki fruit extract, Camellia japonica leaf extract, |
| | | Myrothamnus flabellifolia leaf/stem extract, caprylyl glycol, citric acid, |
| | | ascorbic acid, oat amino acids, wheat amino acids, soy amino acids, |
| | | rice amino acids |
| 7 | Solid | Water, sodium laureth sulfate, sodium lauryl sulfate, cocamidopropyl |
| | foam | betaine, sodium xylenesulfonate, glycol distearate, fragrance, sodium |
| | | citrate, sodium chloride, dimethiconol, piroctone olamine, menthol, |
| | | dimethicone, citric acid, sodium benzoate, guar |
| | | hydroxypropyltrimonium chloride, tea-dodecylbenzenesulfonate, |
| | | tetrasodium edta, sodium hydroxide, trideceth-10, magnesium nitrate, |
| | | methylchloroisothiazolinone, triethylene glycol, propylene glycol, |
| | | benzyl alcohol, magnesium chloride, methylisothiazolinone, copper |
| | | sulfate, blue 1 |

### Combinations

In an aspect, the disclosure relates to a product for reversibly or irreversibly coloring a substrate (e.g., keratinous or textile substrate), the product comprising: a container; a coloring composition in the container, the coloring composition comprising a photochromic compound comprising at least one of: (A) a fluorine-free first compound comprising (1) a cyclic bridge comprising one ethylenically unsaturated bond, (2) a first substituent comprising a first unsaturated heterocycle bonded to a first carbon atom in the ethylenically unsaturated bond and a first aromatic group or a first halogen bonded to the first unsaturated heterocycle, and (3) a second substituent comprising a second unsaturated heterocycle bonded to a second carbon atom in the ethylenically unsaturated bond and a second aromatic group bonded to the second unsaturated heterocycle; (B) a fluorine-free second compound comprising an activated analog of the fluorine-free first compound; and (C) a fluorine-free third compound comprising a dyotropic rearrangement of the fluorine-free first compound (or of the fluorine-free second compound); a light source adapted to provide one or both of activating electromagnetic radiation (EMR) having a wavelength of 250 nm to 395 nm and deactivating EMR having a wavelength of 400 nm to 1400 nm.

In an embodiment, the coloring composition comprises the fluorine-free second compound or the fluorine-free third compound (or activated analog/dyotropic rearrangement of the compound according to Formula (I)).

In an embodiment, the coloring composition comprises the fluorine-free first compound (or compound according to Formula (I)).

In an embodiment, the container is opaque.

In an embodiment, the container is at least partially transparent.

In an embodiment, the light source is adapted to provide activating EMR.

In an embodiment, the light source is adapted to provide deactivating EMR.

In an embodiment, the light source is adapted to provide activating EMR and deactivating EMR.

In an embodiment, the light source is associated with the container interior; and the light source is adapted to irradiate the coloring composition in the container.

In an embodiment, the light source is associated with the container exterior; and the light source is adapted to irradiate the coloring composition external to the container.

In an embodiment, the light source is separate from the container; and the light source is adapted to irradiate the coloring composition external to the container.

In an embodiment, the light source is in the form of a brush or comb.

In an embodiment, the product comprises a first surface comprising the light source; the product comprises a second surface opposing the first surface, the second surface comprising a deposition plate in fluid communication with the container; and the container is adapted to dispense the composition via the deposition plate while being irradiated by the light source.

In an embodiment, the light source and the container containing the coloring composition are spatially separated from each other in a retail environment.

In an embodiment, the light source and the container containing the coloring composition are sold separately.

In an embodiment, the light source and the container containing the coloring composition each display a common brand.

In an embodiment, the coloring composition comprises 0.01 wt. % to 25 wt. % the photochromic compound; and the coloring composition comprises 1 wt. % to 99.99 wt. % of a solvent.

In an embodiment, the coloring composition comprises a plurality of different photochromic compounds; at least one of the plurality of different photochromic compounds comprises at least one of the first fluorine-free compound, the second fluorine-free compound, and the third fluorine-free compound (or at least one of the Formula (I) compounds, analogs, and rearrangements); and optionally, the plurality of different photochromic compounds comprises a compound other than the first fluorine-free compound, second fluorine-free compound, and third fluorine-free compound (or the Formula (I) compounds, analogs, and rearrangements).

In an embodiment, the coloring composition comprises: a first photochromic compound having a first color in activated form; a second photochromic compound having a second color in activated form; and optionally, a third photochromic compound having a third color in activated form.

In an embodiment, each photochromic compound has a characteristic activation wavelength (λₐ) and a characteristic deactivation wavelength (λ_{d}); and for each photochromic compound at least one of its characteristic activation wavelength and characteristic deactivation wavelength is different by at least 60 nm relative to a characteristic activation wavelength or a characteristic deactivation wavelength, respectively, or a different photochromic compound.

In an embodiment, the fluorine-free first compound is a compound according to Formula (I), the fluorine-free second compound is an activated analog of the compound according to Formula (I), and the fluorine-free third compound is a dyotropic rearrangement of the compound according to Formula (I) (or the activated analog thereof).

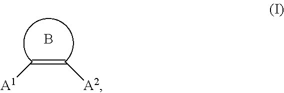

wherein A¹ and A² are independently selected from substituents having Formula (Ia) and substituents having Formula (Ib), with the proviso that at least one of A¹ and A² is a substituent having Formula (Ia):

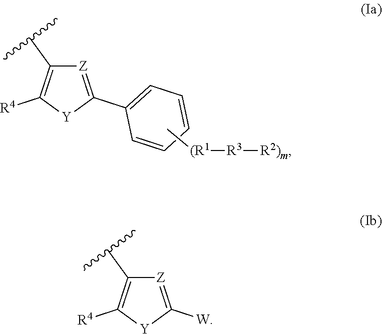

In Formulas (I), (Ia), and/or (Ib), each W is independently selected from Cl, Br, and I; each Y is independently selected from S, O, and NR⁶; each Z is independently selected from N and CR⁷; each R¹ is independently selected from O, S, C═C, C≡C, OC(═O), C(═O)O, C(═O)S, SC(═O), C(═O)NH, NHC(═O), C(═S)NH, NHC(═S), NHC(═O)NH, and NHC(═S)NH; each R² is independently selected from H, C₁₋₆alkyl, N(R⁸)₂, N⁺H(R⁸)₂, and any of the foregoing linked to R³ via an additional R¹ group; each R³ is independently a bond, linear or branched C₁₋₂₀alkylene, or linear or branched (OCH₂CH₂)ₙ, where n is an integer in the range of 1 to 20; each R⁴ is independently selected from C₁₋₈alkyl, C₄₋₈cycloalkyl, C₁₋₁₂alkoxy, C≡CR⁷, and cyano; and each m is independently 1 to 5. Ring B has the formula

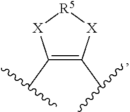

wherein X is CH₂ and R⁵ is a bond or unsubstituted or substituted C₁₋₂ alkylene, or X is carbonyl and R⁵ is O, NH, or NR⁸; wherein each R⁶, R⁷, and R⁸ is independently selected from H, C₁₋₆ alkyl, and aryl.

In an embodiment, the photochromic compound comprises the compound of Formula (I) and the activated analog of the compound according to Formula (I), wherein: a mole ratio of the amount of the compound of Formula (I) to the amount of the activated analog of the compound of Formula (I) is in a range of 1:99 to 99:1, or 5:95 to 95:5, or 10:90 to 90:10, or 25:75 to 75:25; and the compound of Formula (I) is represented by Structure (A) and the activated analog of the compound of Formula (I) is represented by Structure (B), in both of which R is independently selected from W and the substituted phenyl group -[Phenyl]-[R¹-R³-R²]ₘ:

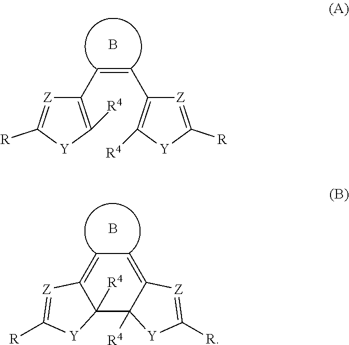

In an embodiment, the photochromic compound comprises the dyotropic rearrangement of the compound according to Formula (I), wherein the dyotropic rearrangement of the compound according to Formula (I) is represented by Structure (C), in which R is independently selected from W and the substituted phenyl group -[Phenyl]-[R¹-R³-R²]ₘ:

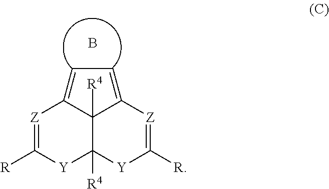

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Throughout the specification, where the compositions, processes, kits, or apparatus are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A product for coloring a substrate, the product comprising:
a container;
a coloring composition in the container, the coloring composition comprising a photochromic compound comprising at least one of:
(A) a fluorine-free first compound comprising (1) a cyclic bridge comprising one ethylenically unsaturated bond, (2) a first substituent comprising a first unsaturated heterocycle bonded to a first carbon atom in the ethylenically unsaturated bond and a first aromatic group or a first halogen bonded to the first unsaturated heterocycle, and (3) a second substituent comprising a second unsaturated heterocycle bonded to a second carbon atom in the ethylenically unsaturated bond and a second aromatic group bonded to the second unsaturated heterocycle;
(B) a fluorine-free second compound comprising an activated analog of the fluorine-free first compound; and
(C) a fluorine-free third compound comprising a dyotropic rearrangement of the fluorine-free first compound;
a light source adapted to provide one or both of activating electromagnetic radiation (EMR) having a wavelength of 250 nm to 395 nm and deactivating EMR having a wavelength of 400 nm to 1400 nm.

2. The product of claim 1, wherein:
the coloring composition comprises the fluorine-free second compound or the fluorine-free third compound.

3. The product of claim 1, wherein:
the coloring composition comprises the fluorine-free first compound.

4. The product of any of claim 1, wherein the container is opaque.

5. The product of any of claim 1, wherein the container is at least partially transparent.

6. The product of any of claim 1, wherein the light source is adapted to provide activating EMR.

7. The product of any of claim 1, wherein the light source is adapted to provide deactivating EMR.

8. The product of any of claim 1, wherein the light source is adapted to provide activating EMR and deactivating EMR.

9. The product of any of claim 1, wherein:
the light source is associated with the container interior; and
the light source is adapted to irradiate the coloring composition in the container.

10. The product of any of claim 1, wherein:
the light source is associated with the container exterior; and
the light source is adapted to irradiate the coloring composition external to the container.

11. The product of any of claim 1, wherein:
the light source is separate from the container; and
the light source is adapted to irradiate the coloring composition external to the container.

12. The product of claim 11, wherein the light source is in the form of a brush or comb.

13. The product of any of claim 1, wherein:
the product comprises a first surface comprising the light source;
the product comprises a second surface opposing the first surface, the second surface comprising a deposition plate in fluid communication with the container; and
the container is adapted to dispense the composition via the deposition plate while being irradiated by the light source.

14. The product of any of claim 1, wherein the light source and the container containing the coloring composition are spatially separated from each other in a retail environment.

15. The product of any of claim 1, wherein the light source and the container containing the coloring composition are sold separately.

16. The product of any of claim 1, wherein the light source and the container containing the coloring composition each display a common brand.

17. The product of any of claim 1, wherein:
the coloring composition comprises 0.01 wt. % to 25 wt. % the photochromic compound; and
the coloring composition comprises 1 wt. % to 99.99 wt. % of a solvent.

18. The product of any of claim 1, wherein:
the coloring composition comprises a plurality of different photochromic compounds;
at least one of the plurality of different photochromic compounds comprises at least one of the first fluorine-free compound, the second fluorine-free compound, and the third fluorine-free compound; and
optionally, the plurality of different photochromic compounds comprises a compound other than the first fluorine-free compound, second fluorine-free compound, and third fluorine-free compound.

19. The product of claim 18, wherein the coloring composition comprises:
a first photochromic compound having a first color in activated form;
a second photochromic compound having a second color in activated form; and
optionally, a third photochromic compound having a third color in activated form.

20. The product of claim 18, wherein:
each photochromic compound has a characteristic activation wavelength (λₐ) and a characteristic deactivation wavelength (λ_{d}); and
for each photochromic compound at least one of its characteristic activation wavelength and characteristic deactivation wavelength is different by at least 60 nm relative to a characteristic activation wavelength or a characteristic deactivation wavelength, respectively, or a different photochromic compound.

21. The product of any of claim 1, wherein the fluorine-free first compound is a compound according to Formula (I), the fluorine-free second compound is an activated analog of the compound according to Formula (I), and the fluorine-free third compound is a dyotropic rearrangement of the compound according to Formula (I) (or the activated analog thereof): 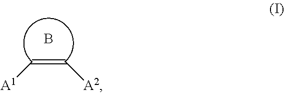
wherein A¹ and A² are independently selected from substituents having Formula (Ia) and substituents having Formula (Ib), with the proviso that at least one of A¹ and A² is a substituent having Formula (Ia): 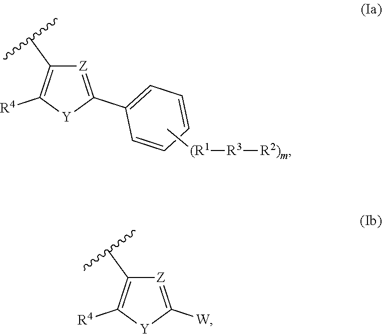
wherein
each W is independently selected from Cl, Br, and I;
each Y is independently selected from S, O, and NR⁶;
each Z is independently selected from N and CR⁷;
each R¹ is independently selected from O, S, C═C, C≡C, OC(═O), C(═O)O, C(═O)S, SC(═O), C(═O)NH, NHC(═O), C(═S)NH, NHC(═S), NHC(═O)NH, and NHC(═S)NH;
each R² is independently selected from H, C₁₋₆alkyl, N(R⁸)₂, N⁺H(R⁸)₂, and any of the foregoing linked to R³ via an additional R¹ group;
each R³ is independently a bond, linear or branched C₁₋₂₀alkylene, or linear or branched (OCH₂CH₂)ₙ, where n is an integer in the range of 1 to 20;
each R⁴ is independently selected from C₁₋₈alkyl, C₄₋₈cycloalkyl, C₁₋₁₂alkoxy, C≡CR⁷, and cyano; and
each m is independently 1 to 5;
wherein ring B has the formula 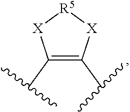
wherein
X is CH₂ and R⁵ is a bond or unsubstituted or substituted C₁₋₂ alkylene, or
X is carbonyl and R⁵ is O, NH, or NR⁸;
wherein each R⁶, R⁷, and R⁸ is independently selected from H, C₁₋₆ alkyl, and aryl.
